(19) United States
(12) Patent Application Publication
(10) Pub. No.: US 2024/0293547 A1
(43) Pub. Date: Sep. 5, 2024

(54) POLYMERIC PERFLUOROCARBON NANOEMULSIONS FOR ULTRASONIC DRUG UNCAGING

(71) Applicant: The Board of Trustees of the Leland Stanford Junior University, Stanford, CA (US)

(72) Inventors: Raag D. Airan, Menlo Park, CA (US); Qian Zhong, Palo Alto, CA (US)

(21) Appl. No.: 18/381,930

(22) Filed: Oct. 19, 2023

Related U.S. Application Data
(62) Division of application No. 16/636,611, filed on Feb. 4, 2020, now abandoned.
(60) Provisional application No. 62/666,417, filed on May 3, 2018.
(60) Provisional application No. 62/545,970, filed on Aug. 15, 2017.

(51) Int. Cl. A61K 41/00; A61B 5/055; A61B 5/16; A61B 5/369; A61K 9/00; A61K 9/107; A61K 31/05; A61K 31/135; A61K 31/277; A61K 31/4174; A61K 31/4422; A61K 31/704; A61K 33/243; A61K 47/34; A61K 49/22; A61M 37/00; A61P 9/08; A61P 23/00

(52) U.S. Cl. CPC A61K 41/0028; A61B 5/055; A61B 5/165; A61B 5/369; A61K 9/0009; A61K 9/0019; A61K 9/0085; A61K 9/1075; A61K 31/05; A61K 31/135; A61K 31/277; A61K 31/4174; A61K 31/4422; A61K 31/704; A61K 33/243; A61K 47/34; A61K 49/226; A61M 37/0092; A61P 9/08; A61P 23/00

(57) ABSTRACT

Disclosed herein are compositions comprising polymeric perfluorocarbon nanoemulsions and methods of their production, as well as methods for their use in imaging, examination, diagnosis and/or treatment of neurological and psychiatric diseases, as well as for ultrasound-mediated localized drug release into the brain.

### CROSS-REFERENCE

This application is a Divisional and claims the benefit of application Ser. No. 16/636,611, filed Feb. 4, 2020, which claims benefit of PCT Application No. PCT/US2018/045783, filed Aug. 8, 2018, which claims benefit of U.S. Provisional Patent Application No. 62/545,970 filed Aug. 15, 2017, and 62/666,417 filed May 3, 2018, each of which applications are incorporated herein by reference in their entirety.

### GOVERNMENT RIGHTS

This invention was made with government support under contracts CA199075 and MH114252 awarded by the National Institutes of Health. The Government has certain rights in the invention.

### TECHNICAL FIELD

The present disclosure generally pertains to medically useful polymeric perfluorocarbon nanoemulsion compositions for ultrasound-gated drug and/or imaging agent release, as well as to methods of making and methods of using said compositions. The compositions and methods disclosed herein are useful as sensors in imaging technologies for assessing brain activity in a subject in vivo, as well as in targeted drug delivery for modulation of brain, heart or other organ function.

### INTRODUCTION

Development of nanoparticles useful in imaging as well as in drug delivery is of great interest; for either purpose, it is desirable to target the nanoparticles to a particular part of the brain or organ in the body.

For example, neuroimaging tools are of clinical and research interest for studying brain function, monitoring spatiotemporal dynamics of brain activities and understanding neural signaling events, as well as for diagnosing neurological diseases or disorders. Functional magnetic resonance imaging MRI (fMRI) is a neuroimaging procedure that measures brain activity in vivo by detecting changes in cerebrovascular blood flow and concomitant changes in neuronal activity. Because fMRI is noninvasive and does not require exposure to ionizing radiation, physicians use fMRI before brain surgery or other invasive treatment for brain mapping, to plan for surgery and radiation therapy. Researchers can also use fMRI to learn how a normal, diseased or injured brain is functioning, and to identify regions linked to critical functions such as speaking/language, memory, moving, sensing, or planning. Clinicians also use fMRI to anatomically map the brain and detect the effects of diseases or trauma, (e.g., stroke, seizures, tumors in the central nervous system (CNS), head and brain injury, pain (including neuropathic pain), Alzheimer's, autism and mood disorders such as depression). Pharmacological fMRI is expected to be useful in measuring brain activity after drugs are administered, to assess how well a drug or behavioral therapy works, and/or to measure drug penetration through the blood-brain barrier and gather dose vs. effect information for a particular medication. Also of great interest to neurological research and medicine are techniques allowing release of a particular pharmacological and/or imaging agent into a specific target area of the brain, for focal modulation of brain function.

Current pre-surgical methods for defining the margin between pathologic and functional brain regions are, primarily, fMRI and the Wada test. The “Wada test” (also known as the intracarotid sodium amobarbital procedure (ISAP), or intracarotid propofol procedure (IPP)) can be used to establish the relative contribution of each cerebral hemisphere to language (speech) and memory functions, and is often used before ablative surgery in patients with epilepsy, and sometimes prior to tumor resection. In a majority of subjects, language (speech) is controlled by the left side of the brain. Though generally considered a safe procedure, there are at least minimal risks associated with the Wada test, as it is an angiography procedure that guides the catheter to the internal carotid artery; thus, researchers are looking into non-invasive ways to determine language and memory laterality—such as fMRI, TMS, magnetoencephalography, and near-infrared spectroscopy.

Other methodologies for scanning the brain include ultrasound-based brain scanning. For example, transcranial ultrasound is used almost exclusively in infants because the soft fontanelle on the skull provides an “acoustic window” for high-frequency sound waves. Useful for patients of any age is Transcranial Doppler (TCD) and Transcranial Color Doppler (TCCD) ultrasonography, which can scan transcranially to measure the velocity of blood flow through the major arteries in the brain.

However, current techniques for brain imaging typically suffer from problems such as lack of precision, low spatial resolution, and difficulties in depth penetration for the ability to access central brain structures. Few good methods are available to noninvasively isolate and study the neurologic and functional anatomy of psychiatric diseases, to specifically isolate and probe peripheral nerves noninvasively, to assess the pharmacological action of drugs in a few isolated brain regions for focal pharmacotherapy, to noninvasively and safely create a reversible ‘pseudo-lesion’ of a brain region, or to evaluate the region prior to medical intervention.

Other shortcomings of current pre-surgical methods employing micro-or nanoparticles include the possibility of embolism if the imaging agent and/or drug-delivery particles introduced into the brain vasculature are too large to pass through vessels and thereby causing a blockage. Present methods of manufacturing often yield a wide range of sizes of the micro-or nanoparticles, increasing the chances of embolism. Furthermore, present methods of manufacturing the particles result in suboptimal levels of loading of the imaging agent and/or drug into the particles, and thus, a large quantity of particles must be administered to achieve an effective dose of the imaging or therapeutic agent. In some cases, particles may be formed from non-biodegradable materials and their action could damage brain tissues. Finally, in some methods, using ultrasound to get particles through the blood-brain barrier (BBB), tissues are actually disrupted with ultrasound waves to allow the agent being delivered to pass through.

The BBB is meant to protect the brain from noxious agents, but, from a research and clinical standpoint, this barrier also significantly hinders the delivery of drugs/imaging agents to the brain. Several strategies have been employed to deliver agents across the BBB, but some of these strategies do structural damage to the BBB by forcibly disrupting/opening it to allow the passage of the desired agent.

A long-felt need remains for compositions and methods for more focused delivery of imaging agents or drugs, as well as for noninvasively mapping the CNS prior to neurosurgery. For example, an ideal method for focused delivery of neurologically acting agents across the BBB should be precisely controlled and should not cause damage to the barrier or the brain itself. Nanotechnology-based delivery methods provide the best prospects for achieving this ideal, and the most useful nanoparticles will be those that can be activated to deliver drug into the living brain, at any depth, with high spatial and temporal precision.

Also desirable is a clinically-translatable platform for production of compositions and methods for noninvasive ultrasonic nanoparticle delivery and uncaging. Such compositions and methods are extremely useful in clinical and research settings, and the present disclosure addresses and overcomes many of the limitations of the presently available compositions and methodologies.

### SUMMARY

Certain aspects, including embodiments, of the present subject matter may be beneficial alone or in combination, with one or more other aspects or embodiments. Without limiting the following detailed description, certain non-limiting aspects of the disclosure are provided below. As will be apparent to those of skill in the art upon reading this disclosure, each of these aspects may be used or combined with any of the preceding or following aspects. This is intended to provide support for all such combinations of aspects and is not limited to combinations of aspects explicitly provided below:

In some aspects, the present disclosure provides a composition comprising a polymeric perfluorocarbon nanoemulsion comprising nanoparticles less than 1 micron in diameter, wherein the nanoparticles comprise (a) an amphiphilic diblock-copolymer; (b) a high vapor pressure liquid core; and (c) a hydrophobic compound selected from a therapeutic agent (drug) and/or a contrast agent.

In some embodiments of the composition or method described herein, the average size of the nanoparticles in the composition is less than 500 nm. In some aspects, the median Z-average diameter of nanoparticles in the nanoemulsion is 400-450 nm.

In some embodiments, the composition further comprises a cryoprotectant. In some embodiments, the cryoprotectant is glycerin. In some embodiments, the cryoprotectant is glycerin at a concentration of 2.25% v/w. In some embodiments, the cryoprotectant is glycerin or sucrose, and is present at a concentration of about 1%, about 1.25%, about 1.5%, about 1.75%, about 2%, about 2.25%, about 2.5%, about 2.75%, or about 3% volume to weight.

In some embodiments of the composition or method described herein, the high vapor pressure liquid core is in a liquid phase before an ultrasound pulse is applied, and the liquid phase changes to a gas phase after the ultrasound pulse is applied. In some embodiments, the liquid core oscillates and/or expands in volume in response to ultrasound. In some embodiments, an ultrasound pulse results in oscillation and/or expansion of the core and release of the hydrophobic compound from the nanoparticles. In some embodiments, the high vapor pressure liquid is a perfluorocarbon. In some embodiments, the high vapor pressure liquid is selected from perfluoromethane, perfluoroethane, perfluoropropane, perfluorobutane, perfluorocyclobutane, perfluropentane, and perfluorohexane.

In some embodiments of the composition or method described herein, the amphiphilic diblock-copolymer comprises a polyethyleneglycol (PEG) complexed with a polymer selected from a polycaprolactone (PCL); a poly(lactide-co-glycolide) (PLGA); and a poly(L-lactic acid) (PLLA).

In some embodiments of the composition or method described herein, the hydrophobic compound is a therapeutic agent. In some embodiments, the hydrophobic compound is a contrast agent. In some embodiments, the hydrophobic compound acts as both a therapeutic agent and a contrast agent. In some embodiments, the hydrophobic compound (i.e., therapeutic and/or contrast agent) is selected from propofol, ketamine, nicardipine, verapamil, dexmedetomidine, modafinil, doxorubicin, and cisplatin. In some embodiments, the therapeutic and/or contrast agent is a drug with log P>1. In some embodiments, the therapeutic agent is a drug with log P>0. In some embodiments, the therapeutic and/or contrast agent is an anesthetic. In some embodiments, the therapeutic and/or contrast agent is a vasodilator. In some embodiments, the composition further comprises an imaging agent and/or dye.

In some aspects, provided herein is a method of producing a polymeric perfluorocarbon nanoemulsion, said method comprising (a) mixing an amphiphilic di-block copolymer and a hydrophobic compound, wherein the hydrophobic compound is selected from a therapeutic agent and a contrast agent, in an organic solvent (e.g., a cyclic ether such as THF, tetrahydropyran, dioxane, dioxolane, etc.); (b) transferring the mixture into normal saline or PBS and, subsequently, evaporating the organic solvent and to produce compound-loaded polymeric micelles; (c) mixing the compound-loaded micelles with a high vapor pressure liquid; (d) sonicating at 40 kHz until the high-vapor pressure liquid is emulsified, forming a compound-loaded nanoemulsion of nanoparticles with a high vapor pressure liquid core; (e) performing membrane extrusion to select for particles under 1 micron; and (f) purifying the polymeric perfluorocarbon nanoemulsion by sequential centrifugation and resuspending in fresh aqueous medium. In some embodiments, steps (e) and (f) are alternated and/or repeated multiple times. In some embodiments, 2.25% v/w glycerin is added after step (f).

In some embodiments of the method, a cryoprotectant such as glycerin or sucrose is present at a concentration of about 1%, about 1.25%, about 1.5%, about 1.75%, about 2%, about 2.25%, about 2.5%, about 2.75%, or about 3% volume to weight.

In some aspects, provided herein is a method of treating or ameliorating a neurological disease or disorder selected from Alzheimer's Disease, epilepsy, tremors, seizures, CNS cancers and tumors (gliomas, glioblastoma multiforme (GBM), medulloblastoma, astrocytoma, diffuse instrinsic pontine glioma (DIPG)), pain, and psychiatric diseases (e.g., PTSD, anxiety disorder, depression, bipolar disease, suicidality), wherein a polymeric perfluorocarbon nanoemulsion composition as described herein is administered intravenously or into the cerebrospinal fluid (CSF) of a subject and an ultrasound pulse is subsequently delivered to the brain or brain vasculature of the subject with an intensity sufficient to yield particle activation.

In some embodiments of the method, the amphiphilic diblock-copolymer (a) is selected from the group consisting of a polycaprolactone (PCL); a poly(lactide-co-glycolide) (PLGA); and a poly(L-lactic acid) (PLLA).

In some embodiments, the composition or method described herein is used in combination with one or more methods of imaging (e.g. fMRI or PET), measuring electrophysiology (e.g. EEG), and/or behavioral assessment of brain function, following focal drug release.

In some aspects of the composition or method described herein, the high vapor pressure liquid core (b) of the nanoparticles in the composition is in a liquid phase before an ultrasound pulse is applied, and the liquid phase changes to a gas phase after the ultrasound pulse is applied. In some aspects of the method, the high vapor pressure liquid core (b) of the nanoparticles in the composition oscillates and/or expands in volume in response to an ultrasound pulse.

In some aspects of the composition or method described herein, the high vapor pressure liquid core (b) of the nanoparticles in the composition is a perfluorocarbon. In some aspects, the high vapor pressure liquid is selected from perfluoromethane, perfluoroethane, perfluoropropane, perfluorobutane, perfluorocyclobutane, perfluropentane (PFP), and perfluorohexane.

In some aspects, a neurally-active/neuromodulator drug is used as a therapeutic agent, and is selected from propofol, ketamine, nicardipine, verapamil, dexmedetomidine, modafinil, doxorubicin, and cisplatin. In some embodiments, the hydrophobic compound is a therapeutic agent. In some embodiments, the therapeutic agent is a vasodilator.

For glioblastomas, chemotherapy with temozolomide is now routinely given with radiation therapy. The dose is 75/mg/m²/day (including weekend days when radiation is skipped) for 42 days, then 150 mg/m² po once/day for 5 days/mo during the next month, followed by 200 mg/m² po once/day for five days/mo in subsequent months for a total of 6 to 12 mo. During treatment with temozolomide, trimethoprim/sulfamethoxazole 800 mg/160 mg is given three times/wk to prevent Pneumocystis jirovecii pneumonia. For medulloblastomas, drugs include nitrosoureas, procarbazine, vincristine alone or in combination, intrathecal methotrexate, combination chemotherapy (e.g., mechlorethamine, vincristine [Oncovin], procarbazine, plus prednisone [MOPP]), cisplatin, and carboplatin).

In some aspects of the method, the composition further comprises an imaging agent and/or dye.

In some aspects, provided herein is a method of producing a polymeric perfluorocarbon nanoemulsion, said method comprising (a) mixing an amphiphilic diblock-copolymer and a hydrophobic compound selected from a therapeutic agent/drug and a contrast agent in an organic solvent (e.g., a cyclic ether such as tetrahydrofuran (THF), etc.); (b) transferring the mixture into an aqueous medium (e.g. normal saline or Phosphate Buffered Saline (PBS), etc.) and, subsequently, evaporating the organic solvent to produce compound-loaded polymeric micelles; (c) mixing the compound-loaded micelles with a high vapor pressure liquid; (d) sonicating at 40 kHz for typically 3-5 min, up to 15 min. to emulsify the high-vapor pressure liquid and form a compound-loaded nanoemulsion of nanoparticles with a high vapor pressure liquid core; (e) performing membrane extrusion to select for particles under 1 micron; and (f) purifying the nanoparticles by sequential centrifugation and resuspending in fresh aqueous medium. In some embodiments, steps (e) and (f) are alternated and repeated to optimally hone the size range of the resultant nanoparticles and reduce particle aggregation.

In some aspects, provided herein is a method of treating or ameliorating a neurological disease or disorder selected from Alzheimer's Disease, epilepsy, tremors, seizures, CNS cancers and tumors (gliomas, glioblastoma multiforme (GBM), medulloblastoma, astrocytoma, diffuse instrinsic pontine glioma (DIPG)), pain (including neuropathic pain), and psychiatric diseases (e.g., PTSD, anxiety disorder, depression, bipolar disease, suicidality), wherein a polymeric perfluorocarbon nanoemulsion composition is administered intravenously or into the cerebrospinal fluid (CSF) and an ultrasound pulse is subsequently delivered to the brain or brain vasculature of a subject, with an intensity sufficient to yield particle activation and using sonication parameters sufficient to induce particle activation (e.g. sonication at 1 MHZ, inducing a peak negative pressure of 1.0 or 1.5 MPa, for 50 milliseconds (ms), repeated at 1 Hz×60 seconds). In some embodiments, the pressure is between 0.8 and 1.8 MPa, at a burst length of 10-100 ms. In some embodiments, the ultrasound frequency is between 0.2 and 2.0 MHz.

In some aspects, provided herein is a method of treating or ameliorating a cardiovascular disease or disorder selected from, for example, hypertension, arterial spasm or blockage, cerebral vasospasm, and myocardial or other end organ infarction or ischemia, wherein the polymeric perfluorocarbon nanoemulsion composition described herein is administered intravenously and an ultrasound pulse is subsequently delivered to a localized cardiovascular region in the subject with an intensity sufficient to yield particle activation.

These and other objects, advantages, and features of the disclosure will become apparent to those persons skilled in the art upon reading the details of the compositions and methods as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-1G: present a schematic showing production of perfluoropentane nanoparticles for ultrasonic drug uncaging, and comparisons of their stability, Z-average diameter, and drug loading characteristics.

FIG. 2A-2D: compare Z-average diameter, polydispersity index, drug loading, and ultrasonic uncaging characteristics of various polymer choices for drug-loaded perfluoropentane nanoemulsions.

FIG. 3A-3D: compare Z-average diameter, polydispersity index, compound loading, and ultrasonic drug uncaging of various hydrophobic drugs.

FIG. 4A-4D: depict the particle clearance kinetics, biodistribution, and biotolerance of FIG. 4A propofol-loaded nanoparticles (bolus of 1 mg/kg encapsulated propofol), FIG. 4B propofol-loaded nanoparticles as an i.v. infusion (bolus of 1 mg/kg+infusion of 1.5 mg/kg/hr encapsulated propofol), FIG. 4C nicardipine-loaded nanoparticles, and FIG. 4D doxorubicin-loaded nanoparticles.

FIG. 5A-5C: show sample images of IR dye fluorescence and tissue distribution of propofol, nicardipine, or doxorubicin-loaded nanoparticles in rats.

FIG. 6A-6D: illustrate that ultrasonic propofol uncaging reversibly anesthetizes the visual cortex.

FIG. 7A-7F: demonstrate that ultrasonic uncaging of nicardipine-loaded nanoparticles locally increases aortic wall compliance in vitro and in vivo.

FIG. 8A-8F: show two-phase decay and one-phase decay modeling of blood-pool kinetics of nanoemulsions after bolus administration. FIGS. 8A and 8B: Propofol-loaded nanoemulsions. FIGS. 8C and 8D: Nicardipine-loaded nanoemulsions. FIGS. 8E and 8F: Doxorubicin-loaded nanoemulsions.

### DETAILED DESCRIPTION

Ultrasound-mediated drug delivery has gained much attention recently with the availability of clinical focused ultrasound systems that may sonicate any region of the body with millimeter spatial resolution. These technologies may use nano-or micro-scale drug carriers that release drug after ultrasound raises the in situ temperature, activates a ‘sonosensitizer’, or raises the tissue intensity/pressure to beyond a certain threshold. While high-intensity continuous wave ultrasound may be difficult to achieve stably in certain regions of the body, the intensity/pressure uncaged systems usually necessitate only short bursts of ultrasound that are more straightforward to implement in situ.

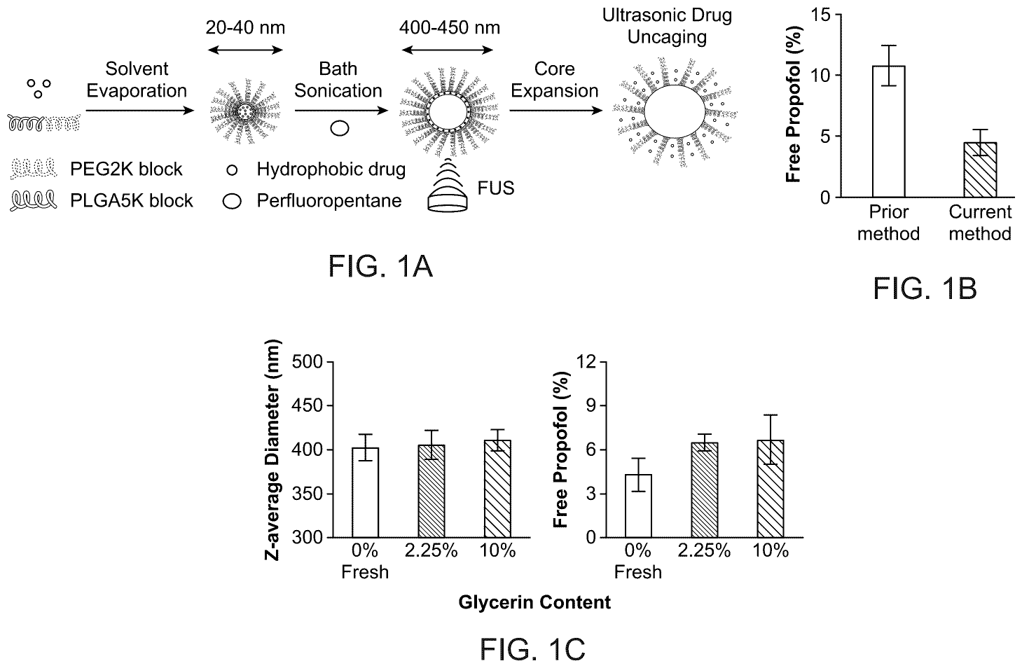

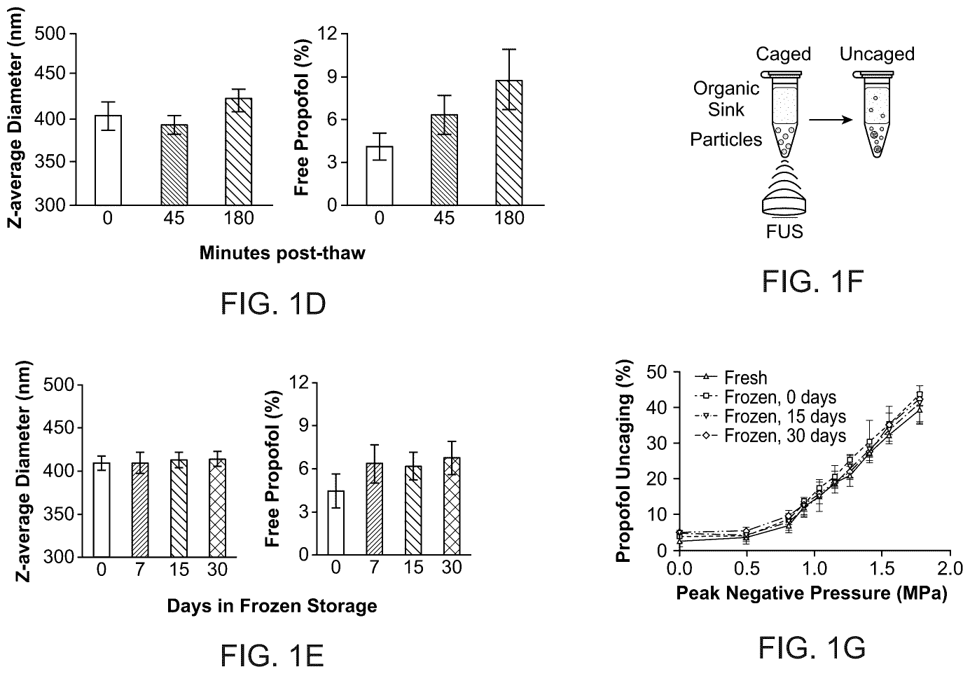

For example, presented herein is a polymeric perfluoropentane nanoemulsion that can locally uncage the anesthetic propofol in the brain with short bursts of focused ultrasound (FUS), thereby enabling noninvasive pharmacologic neuromodulation (Airan, R. D. et al., (2017) Nano Lett. 17, 652-659; Airan, R. (2017) Science 357:465). In some embodiments, the nanoparticles described herein are composed of a nanoscale droplet of the high-vapor pressure liquid perfluoropentane (PFP), with drug bound by an emulsifying amphiphilic diblock-copolymer. Without being limited by theory, it is believed that the drug is bound by the hydrophobic polymer block, which sits between the hydrophilic block externally and the core perfluorocarbon internally, with the external hydrophilic block insulating the drug from exposure to the medium. Upon exposure to focused ultrasound of a sufficient peak negative pressure, the core PFP of these particles expands, thinning the emulsifying polymer layer, which exposes the drug to the medium, allowing drug release (FIG. 1a).

These nanoparticles are useful for imaging, oxygen delivery for ischemia, micro-embolization, thermal ablation, blood-brain barrier opening, and as drug delivery vehicles, such as for delivery of chemotherapeutics and/or propofol. These nanoparticles allow encapsulation of any small molecule that is hydrophobic and therefore able to be stably bound by the internal hydrophobic polymer block. The range of drug and polymer characteristics that allow encapsulation into these nanoparticles is systematically described herein. Furthermore, the present compositions and methods meet the demands of clinical manufacturing methods, as they are practically feasible, scalable, compatible with current good manufacturing practices (cGMP), and produce particles that are sufficiently stable for a variety of uses and for storage. In some embodiments of the composition and methods of its manufacture, a cryoprotectant(s) is added (e.g. glycerin or sucrose), as cryoprotectants are demonstrated herein to dramatically improve nanoparticle stability, allowing the nanoparticles to survive one or more freeze-thaw cycles. Thus, the presently disclosed polymeric perfluorocarbon nanoemulsion composition comprising nanoparticles has improved long-term storage characteristics, which also allows manufacture and distribution from a central production facility. Also presented herein is an explicit demonstration of the in vivo efficacy of this system in different regions of the body and on different organ systems. The presently described compositions and methods provide a versatile platform for ultrasonic uncaging of a variety of drugs, and enable translation into the clinical setting.

The polymeric perfluorocarbon nanoemulsion compositions and methods described herein are useful for in vivo imaging of a specifically targeted organ or structure (e.g., a particular region of the brain, structure in the heart, alveoli of the lungs, etc.) in a subject, as well as for administering an effective amount of a therapeutic agent to a particular organ (for example, the heart, or brain, brain vasculature, lungs and/or alveoli, etc.) in a patient in vivo, then uncaging the agent by applying a targeted ultrasound pulse, in order to administer the agent to a highly focalized region.

Described herein are protocols that result in consistent nanoparticle size, monodispersity, drug loading, and stability, and which hew to clinical production standards. The nanoparticle compositions and systems provided herein allow targeted delivery and uncaging of a variety of drugs or imaging agents at a desired time and place using focused ultrasound. The in vivo efficacy of the compositions and methods is demonstrated in two organ systems: first, targeted modulation of brain activity with anesthetic uncaging is demonstrated, and second, local control of cardiovascular function upon vasodilator uncaging is demonstrated.

The nanotechnology described herein provides a robust and spatiotemporally precise, noninvasive technique for pre-surgical brain mapping and imaging brain function (in some ways similar to the Wada test), as well as for highly localized (focal) release of a nanoparticle-encapsulated drug and/or imaging agent into a specific region of the central and/or peripheral nervous system using focused ultrasound (FUS). The system can encapsulate and deliver most any small molecule drug, especially lipophilic drugs that would normally cross the blood-brain barrier. The system is effective, safe, and that the particles can be scaled up for large scale production using cGMP-compatible methods.

In addition to brain imaging and presurgical mapping of functional brain regions to identify a surgical tract between critically important brain regions and a lesion to be resected and a margin around the lesion, the compositions and methods described herein are useful in basic research and clinical applications in psychiatry. Some other applications for the compositions and methods described herein include pre-hoc validation of a brain region to be intervened upon with, for example, deep brain stimulation (DBS), radiosurgery, radiofrequency ablation (RFA), laser ablation, or focused ultrasound (FUS) ablation.

For example, the compositions and methods described herein can be used for validating the location of the ventral intermediate (VIM) thalamic nucleus prior to ablation for essential tremor or tremor-dominant Parkinson disease (PD), or for validating a focus as a principal seizure generator prior to resection or ablation.

Another application for the compositions and methods described herein is adjunctive focal pharmacotherapy for psychiatric treatment; for example, modulating processes in the amygdala in real time using anti-adrenergic therapeutic agents during talk or exposure therapy sessions for PTSD or anxiety disorder. Alternatively, ketamine may be infused locally into the ventromedial prefrontal cortex (vmPFC) of an acutely depressed or suicidal patient in order to isolate ketamine's antidepressant action over its anesthetic, addictive, and psychotogenic actions. Similarly, the compositions and methods described herein can be used in focused delivery of epileptogenic treatments or to focally decrease activity of a pathologic neural circuit.

Another application for the compositions and methods described herein is to determine which peripheral nerves most contribute to a complex regional pain syndrome through sequential anesthesia of each nerve, or to ablate certain targets, wherein the composition comprising the nanoparticles described herein can be used for thermal ablation via super-heating at the sonication focus.

A basic research application for the compositions and methods described herein is for validating/testing a hypothesis of the role of a brain region in the performance of a particular brain function, or a receptor's action in a specific brain region (e.g. validation of insular subfields as necessary for certain risk calculations in decision making).

Another application for the compositions and methods described herein is for focal delivery of vasoactive substances to treat alterations of perfusion, e.g. focally delivering calcium channel antagonists like verapamil and/or nicardipine to treat cerebrovascular disorders such as stroke, cerebral vasospasm, or reversible cerebral vasoconstriction syndrome (RCVS).

Another application for the compositions and methods described herein is for the focal delivery of therapeutic agents to treat a cardiovascular disease or disorder selected from hypertension, arterial spasm or blockage, cerebral vasospasm, and myocardial or other end organ infarction or ischemia.

These techniques for exquisitely focalized delivery of a therapeutic agent/drug and/or imaging agent to an organ or organ substructure, such as the cardiovascular system, heart, blood vessels, brain or brain vasculature, lungs and/or alveoli, for example, can transform both basic research and clinical science. The polymeric perfluorocarbon nanoemulsions described herein have been adapted for encapsulation and focal delivery of brain-active drug and/or imaging agents using focused ultrasound (FUS). Nanoparticles smaller than 1 micron for efficient and highly localized delivery of such agents to specific locations in the brain and blood vessels of the brain can be produced with scalable and cGMP-compatible methods. The particles consist of a high-vapor pressure liquid core, emulsified by a block copolymer, having a drug bound internally. To produce the phase-change nanoparticles, the emulsifying amphiphilic diblock-copolymer and the hydrophobic compound selected from a therapeutic agent/drug and a contrast agent are dissolved in an organic solvent (e.g., THF), and transferred to an aqueous medium (e.g., saline/PBS). The organic solvent is then evaporated, leaving behind micelles of the polymer and drug suspended in the aqueous medium. The high vapor pressure liquid is then added and the mixture is sonicated in a bath sonicator until a compound-loaded nanoemulsion of nanoparticles with a high vapor pressure liquid core is formed. The resultant nanoparticles are then extruded through a membrane to select for particles under 1 micron, and further purified by sequential centrifugation and resuspension in fresh aqueous solution. The membrane extrusion and centrifiguation steps may be alternated to select the ideal size range of the nanoparticles or to minimize particle aggregation. The nanoparticles so formed are amenable to sonication at intensities achievable by the FUS transducer and safe for human applications.

There are several advantages to the present polymeric perfluorocarbon nanoemulsion compositions and methods over any of similar compositions and methods previously described. For example, it is herein observed that a step employing bath sonication yields several advantages when emulsifying the nanoparticles. First, a bath sonicator produces waves originating from the undersurface of the container which may be resting in an ice bath, allowing production of more evenly sized particles with significantly better drug-loading (reduction in free drug not encapsulated into the nanoparticles). Second, a bath sonication eliminates one problem common in other manufacturing methods, which is that probe sonication is not a sterile process. A bath sonication removes this non-sterile step, because the container holding the composition to be bath sonicated can be autoclaved, and the composition to be sonicated can be enclosed with a lid, to keep it sterile during the sonication process.

The presently described compositions and methods are particularly useful in clinical settings for focal drug-delivery, as they are easily adapted to be specific for the encapsulation and delivery of a wide range of brain-active neuromodulating agents and accounting for certain chemical features of the drug. The drug is not released generally into the brain or brain vasculature until after a FUS pulse uncages and releases the drug to a very defined region of the brain. Thus, specific noninvasive neuromodulation can be achieved. Another advantage of the present methods of manufacture of the polymeric perfluorocarbon nanoemulsion compositions is that the present method is quite amenable to large-scale cGMP production.

Using the compositions and methods described herein, a FUS-pulse-mediated focal drug release into the brain or brain vasculature can be achieved. The nanoparticles manufactured by the methods described herein are of a size large enough to be restricted from passing through BBB before the FUS pulse, but after the FUS pulse is applied (and, without being bound by theory, after the particle activation occurs), the encapsulated agent/drug is small enough to pass through the BBB. The activated nanoparticle size is limited by physical limitations related to the ideal gas law so that they are smaller than a capillary diameter, and thus, the nanoparticles of the present disclosure reduce the risk of embolism that has been observed when larger nano-or micro-scale particles are used.

Also contemplated is the introduction of the compositions of the present disclosure into the lymphatic system.

The present disclosure addresses and overcomes several problems with and limitations of other technologies. For example, the presently described method of manufacture provides a much improved uniformity of size of nanoparticles (as measured by the polydispersity index), as well as a greater temperature and time stability, and the loading efficiency of the agent encapsulated is doubled. For example of the latter, other groups see significant amounts of free drug/agent that is not loaded into nanoparticles at the zero time point before the FUS pulse. Improved drug-loading with less free drug means that a smaller amount of particles overall can be delivered before the pulse of ultrasound, to deliver a focally effective drug amount, while minimizing potential issues of drug toxicity or overexposure to the drug/agent outside the focal region of the brain one desires to treat, and minimizing the potential for systemic side-effects.

### Definitions

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, some potential and preferred methods and materials are now described. All patents, patent applications and non-patent publications mentioned herein are incorporated herein by reference in their entirety to disclose and describe the methods and/or materials in connection with which the publications are cited. It is understood that the present disclosure supercedes any disclosure of an incorporated publication to the extent there is a contradiction.

Where a range of values is provided, it is understood that each intervening value, to the tenth of the unit of the lower limit unless the context clearly dictates otherwise, between the upper and lower limits of that range and any other stated or intervening value in that stated range, is encompassed and specifically disclosed. Each smaller range between any stated value or intervening value in a stated range and any other stated or intervening value in that stated range is encompassed within the present disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

It must be noted that as used herein and in the appended claims, the singular forms “a,” “an,” and “the” include plural referents unless the context clearly dictates otherwise. Thus, for example, reference to “a nanoparticle” includes a plurality of such nanoparticles, and reference to “the therapeutic agent” includes reference to one or more therapeutic agents and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Furthermore, it is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The well-known “Wada test” (also known as the intracarotid sodium amobarbital procedure (ISAP)) is used to establish the relative contribution of each cerebral hemisphere to language (speech) and memory functions, and is often used before ablative surgery in patients with epilepsy, and sometimes prior to tumor resection. In a majority of subjects, language (speech) is controlled by the left side of the brain. Though generally considered a safe procedure, there are at least minimal risks associated with the angiography procedure that guides the catheter to the internal carotid artery, and thus, researchers are looking into non-invasive ways to determine language and memory laterality—such as fMRI, TMS, magnetoencephalography, and near-infrared spectroscopy.

Other publications have described: a biomembrane phase-change microparticle with a hydrophobic liquid core that is vaporized by ultrasonic radiation (See U.S. Patent Application Publication 20160317441A1), and a nanoparticle having a phase-change material as its inner core and configured to absorb heat (See PCT Publication WO 2016/084082A1).

Drug-loaded perfluorocarbon nanodroplets for ultrasound-mediated drug delivery have been described (Rapoport, N. (2016) Advances in Experimental Medicine and Biology, vol. 880:221-241; U.S. Pat. No. 8,709,451).

The blood-brain barrier (BBB) is a system of vascular structures, enzymes, receptors and transporters designed to prevent access of potentially toxic molecules into the CNS, and to enable passage of nutrients, such as glucose, into brain tissues/structures. The continuous capillaries forming the BBB are sealed and have no fenestrations (openings), forming special tight junctions that restrict paracellular transport. Molecules are restricted from passing between the adjacent cells in capillaries of the CNS by these tight junctions, and pinocytosis is also limited across these capillaries; thus, the main mechanism by which molecules/drugs/imaging agents can pass through the capillaries of the CNS into the brain is passive transcellular diffusion. The molecules transported by passive transcellular diffusion are limited to low molecular weight lipophilic molecules, and this permeability of the BBB is proportional to the lipophilicity of the low molecular weight molecules. However, above a certain molecular weight, the permeability of lipophilic molecules across the BBB is substantially reduced.

Compared with the vasculature of many other organs, the normal BBB severely restricts the passage of most drugs from plasma to the extracellular space, with more than an 8-log difference in the entry rate of small, lipid-soluble molecules compared with large proteins. A few macromolecules are able to enter the brain tissue from the blood by a receptor-mediated process; for example, brain cells require a constant supply of iron to maintain their function and the brain may substitute its iron through transcytosis of iron-loaded transferrin (Tf) across the brain microvasculature. Other biologically active proteins, such as insulin and immunoglobulin G, are actively transcytosed through BBB endothelial cells. The presence of receptors involved in the transcytosis of ligands from the blood to the brain offers opportunities for developing new approaches to the delivery of therapeutic compounds across the BBB (Jain, K., (2012) Nanomedicine. 7(8):1225-1233).

Several strategies have been used for manipulating the BBB for drug delivery to the brain, including osmotic and chemical opening of the BBB as well as the use of transport/carriers. However, the drawbacks of such strategies to forcibly open the BBB include causing damage to the barrier and/or allowing uncontrolled passage of drugs or other noxious agents into the brain. Bypassing the BBB by an alternative route of delivery such as transnasal delivery may also be considered. If targeted delivery to brain parenchyma is not the goal, alternative methods for crossing the blood-cerebrospinal fluid barrier may be considered or drugs may be introduced directly in the cerebrospinal fluid pathways by lumbar puncture. Invasive procedures for bypassing the BBB include direct introduction in the brain by surgical procedures. Several potentially effective therapeutic agents for neurological disorders are available but their use is limited because of insufficient delivery across the BBB (Jain, K., (2012) Nanomedicine. 7(8):1225-1233).

The upper limit of pore size in the BBB that enables passive flow of molecules across it is usually<1 nm; however, particles that have a diameter of several nanometers can also cross the BBB by carrier-mediated transport. Thus, although very small nanoparticles may sluggishly pass through the BBB, this uncontrolled passage into the brain may not be desirable and strategies are being developed for controlled passage as well as targeted drug delivery to the brain (Jain, K., (2012) Nanomedicine. 7(8):1225-1233).

Nanoparticles larger than a few nanometers are not allowed passage through the BBB into brain tissue. Neurologically acting compounds are sometimes modified physically or chemically to allow them to pass from the blood stream into the cranium. As noted above, another solution to administering neurologically acting compounds is to increase permeability of the BBB using receptor-mediated permabilizer compounds. These compounds increase the permeability of the blood-brain barrier temporarily by increasing the osmotic pressure in the blood which loosens the tight junctions between the endothelial cells. By loosening the tight junctions, injection of compositions through an IV can take place and be effective to enter the brain.

Herein, polymeric perfluoropentane nanoemulsions are shown to be a generalized platform for targeted drug delivery with high potential for clinical translation.

In some embodiments, the compositions disclosed herein comprise a polymeric perfluorocarbon nanoemulsion comprising nanoparticles which can cross the blood brain barrier. In some embodiments, the compositions disclosed herein comprise nanoparticles which, before treating the subject with transcranial FUS, cannot cross the blood brain barrier (BBB), but which, upon treating with FUS, uncage a lipophilic drug or imaging agent that can cross the BBB.

In some embodiments of the method of manufacturing the polymeric perfluorocarbon nanoemulsions, compound-loaded polymeric micelles are formed using a sonicator. In some embodiments, the compound-loaded polymeric micelles are mixed with a high vapor pressure liquid. In some embodiments, a sonication step is performed at 40 kHz to form a compound-loaded nanoemulsion of nanoparticles with a high vapor pressure liquid core. In some embodiments, a sonication is performed within the range of above 20 KHz but below 100 KHz.

Medicinal and/or pharmaceutical agents useful in the presently disclosed compositions and methods may have psychoactive, neuromodulating, anaesthetic, analgesic, anti-inflammatory, anti-proliferative, or vasoactive properties.

The nanoparticles used in the methods described herein are biodegradable, do not cause embolism or otherwise damage brain tissues, as has been observed with other FUS-mediated technologies that physically disrupt the BBB to allow the agent's passage through the barrier. Such methods employing FUS to increase permeability by causing interference in the tight junctions and disrupting the BBB in localized areas of the brain allowing extravasation of the agent are described in U.S. Patent Application US 2009/0005711, U.S. Pat. Nos. 6,514,221, and 7,344,509, each of which is hereby incorporated in its entirety.

As used herein, “polydispersity index” (PDI) is defined as the ratio of weight average molecular mass (MW) to the number average molecular mass (Mn), and represented by the equation: PDI=MW/Mn. The ratio of the two can be used to describe how far away the encountered distribution is from a uniform distribution. Thus, PDI is used to describe the degree of homogeneity or non-uniformity of a distribution (herein, a population of nanoparticles). For a perfectly uniform (“monodisperse”) sample consisting of exactly one and only one molecular weight both the Mw and the Mn would be the same value. For synthetic molecules/particles, however, the two numbers are not the same, and MW is usually greater than Mn, and therefore the PDI is greater than one. The larger the polydispersity index, the broader the molecular weight range. In calculating PDI of nanoparticles in a distribution of nanoparticles made by the methods described herein, for example, MW describes their average molecular weight by mass and Mn describes their average molecular weight by number. Mn may be determined by employing methods which depend upon the number of molecules present in the polymer sample. For example, colligative property such as osmotic pressure is used. Weight average molecular mass (MW) may be measured using methods such as light scattering and ultracentrifugation, sedimentation, etc. which depend upon the mass of individual molecules.

In certain instances, following generation of the nanoparticles, they may be size selected, isolated and/or purified according to any convenient method known for isolation and/or purification of nanoparticles. Thus, the isolated and purified nanoparticles may be delivered to a subject unprocessed or they may be size selected, isolated and/or purified by any convenient method described herein or known in the art. The methods of manufacturing the nanoparticles of the present disclosure may involve purification steps, such as membrane extrusion to select for nanoparticles under 1 micron, and/or to select for nanoparticles under 500 nM, and/or to select for nanoparticles under 250 nM. Purification may additionally or alternatively be performed using sequential centrifugation and resuspension in fresh aqueous solution.

In some embodiments, the composition comprises nanoparticles that are substantially spherical. The nanoparticles of the present disclosure may have an average diameter of about 1 micron (1000 nm) or less, about 700 nm or less, about 600 nm or less, about 500 nm or less, about 400 nm or less, about 350 nm or less, about 300 nm or less, about 250 nm or less, about 200 nm or less, about 150 nm or less, or about 100 nm or less. The nanoparticles of the present disclosure preferably have an average diameter of between about 10 nm and about 1 micron (1000 nm), between about 10 nm and about 700 nm, between about 10 nm and about 600 nm, between about 10 nm and about 500 nm, between about 10 nm and about 400 nm, between about 10 nm and about 350 nm, between about 10 nm and about 300 nm, between about 10 nm and about 250 nm, between about 10 nm and about 200 nm, between about 10 nm and about 150 nm, or between about 10 nm and about 100 nm.

In some embodiments, the polymeric perfluorocarbon nanoemulsions of the present disclosure comprise biodegradable polymeric materials. In some embodiments, the polymeric perfluorocarbon nanoemulsion comprising nanoparticles of the present disclosure comprises amphiphilic diblock-copolymers. Exemplary block copolymers include:
polyethylene glycol-poly(lactic-co-glycolic acid) (PEG2k-PLGA2k), MW: PEG=2 kDa and PLGA=2 kDa;
polyethylene glycol-poly(lactic-co-glycolic acid) (PEG2k-PLGA5k), MW: PEG=2 kDa and PLGA=5 kDa;
polyethylene glycol-poly(ε-caprolactone) (PEG2k-PCL2k), MW: PEG=2 kDa and PCL=2 kDa;
polyethylene glycol-poly(ε-caprolactone) (PEG2k-PCL5k), MW: PEG=2 kDa and PCL=5 kDa;
polyethylene glycol-poly(L-lactic acid) (PEG2k-PLLA2k), MW: PEG=2 kDa and PLLA=2 kDa;
polyethylene glycol-poly(L-lactic acid) (PEG2k-PLLA5k), MW: PEG=2 kDa and PLLA=5 kDa.

In some embodiments, an effective amount of a composition disclosed herein is administered to the subject, and a magnetic resonance image (MRI) of the subject's brain is obtained by imaging the target compound.

In some embodiments, the methods disclosed herein for focal drug release can be combined with methods of imaging (e.g. fMRI), methods of measuring electrophysiology (e.g. EEG), or methods of behavioral assessment of brain function, following focal drug release.

In some embodiments, the polymeric perfluorocarbon nanoemulsion of the present disclosure comprises a contrast agent and/or a therapeutic agent/drug selected from propofol, ketamine, nicardipine, verapamil, dexmedetomidine, modafinil, doxorubicin, and cisplatin. In some embodiments, the therapeutic agent is propofol.

In some embodiments, the polymeric perfluorocarbon nanoemulsions of the present disclosure comprise a high vapor pressure liquid in the core of the nanoparticle. In some embodiments, the high vapor pressure liquid is an organic hydrocarbon that is in a liquid phase between approximately 25 oC and 36 oC in 1 atm. In some embodiments, the high vapor pressure liquid in the nanoparticle core is the drug being delivered. In some embodiments, the high vapor pressure liquid is a volatile anaesthetic (e.g., isofluorane, ether, halothane). In some embodiments, the high vapor pressure liquid is a perfluorocarbon (e.g., perfluoromethane, perfluoroethane, perfluoropropane, perfluorobutane, perfluorocyclobutane, perfluoropentane, or perfluorohexane). In some embodiments, the high vapor pressure liquid is perfluoropentane.

Without being bound by theory, in some embodiments, the high vapor pressure liquid in the core of the nanoparticle is a liquid that mediates the particle activation and lowers the threshold and lessens the amount of ultrasound energy to be deposited. For example, perfluorobutane would be used instead of perfluoropentane as the boiling point of perfluorobutane is lower, yielding a lower threshold of sonication intensity for particle activation In some embodiments, the high vapor pressure liquid in the core of the nanoparticle is a liquid that mediates the particle activation and increases the threshold amount of ultrasound energy to improve specificity of uncaging and release of the agent to a specific region of the brain. For example, perfluoropentane has a higher boiling point than perfluorobutane, meaning a higher sonication intensity would be needed for particle activation, and therefore lower risk of nonspecific, spontaneous, or off-target activation.

Without being bound by theory, in some embodiments, the ultrasound pulse induces an overall oscillation of the core of the nanoparticles and concomitant expansion/oscillation of the polymer layer, inducing release of the hydrophobic compound (i.e., therapeutic or contrast agent) from the nanoparticles via an expansion of the core.

A “fluorophore” is a molecule that absorbs light at a characteristic wavelength and then re-emits the light most typically at a characteristic different wavelength. Fluorophores are well known to those of skill in the art and include, but are not limited to rhodamine and rhodamine derivatives, fluorescein and fluorescein derivatives, coumarins and chelators with the lanthanide ion series. A fluorophore is distinguished from a chromophore which absorbs, but does not characteristically re-emit light. “Fluorophore” refers to a molecule that, when excited with light having a selected wavelength, emits light of a different wavelength, which may emit light immediately or with a delay after excitation. Fluorophores, include, without limitation, fluorescein dyes, e.g., 5-carboxyfluorescein (5-FAM), 6-carboxyfluorescein (6-FAM), 2′,4′,1,4,-tetrachlorofluorescein (TET), 2′,4′,5′,7′,1,4-hexachlorofluorescein (HEX), and 2′,7′-dimethoxy-4′,5′-dichloro-6-carboxyfluorescein (JOE); cyanine dyes, e.g. Cy3, CY5, Cy5.5, etc.; dansyl derivatives; 6-carboxytetramethylrhodamine (TAMRA), BODIPY fluorophores, tetrapropano-6-carboxyrhodamine (ROX), ALEXA dyes, Oregon Green, and the like. Combinations of fluorophores also find use, e.g. where transfer or release of a fluorophore leads to a color change.

In some embodiments, the agent encapsulated within the polymeric perfluorocarbon nanoemulsion comprising nanoparticles is a fluorophore. In some embodiments, the fluorophore is propofol. In some embodiments, the agent encapsulated within the polymeric perfluorocarbon nanoemulsion comprising nanoparticles is a macromolecule such as an antibody or antibody fragment or a peptide. In some embodiments, the agent encapsulated within the nanoparticles is a small molecule drug. In some embodiments, the small molecule drug has a Log P greater than 0 and is hydrophobic. For example, propofol has a log P of 3.79, ketamine has a log P of 2.18, doxorubicin has a log P of 1.27. In some embodiments, the agent encapsulated within the nanoparticles of the present disclosure is a contrast or imaging agent for imaging of the brain or brain vasculature. In some embodiments, the contrast or imaging agent is a dye. In some embodiments, the contrast or imaging agent is a fluorophore. In some embodiments, the contrast or imaging agent is selected from gadolinium-containing compounds, iodine-containing compounds, and superparamagnetic iron oxide.

The compositions disclosed herein may comprise contrast agents to enhance contrast in MRI or fMRI, as well as may be used for analyte detection. The early and widely implemented MRI contrast agents are small-molecule chelates that incorporate paramagnetic ions that alter T1, such as gadolinium (Gd3+) or manganese (Mn2+or Mn3+). In some embodiments, the contrast agent may comprise gadolinium (Gd). Non-limiting examples of Gd-comprising contrast agents are gadoterate, adodiamide, gadobenate, gadopentetate, gadoteridol, gadoversetamide, gadoxetate, gadobutrol, gadoterate, gadodiamide, gadobenate, gadopentetate, gadoteridol, gadofosveset, gadoversetamide, gadoxetate, and gadobutrol. In some embodiments, the contrast agent comprises 1,4,7,10-tetraazacyclododecane-1,4,7,10-tetraacetic acid (DOTA). In other embodiments, the contrast agent is DOTA-Gd. The contrast agent may be GdNP-DO3A (gadolinium 1-methlyene-(p-NitroPhenol)-1,4,7,10-tetraazacycloDOdecane-4,7,10-triAcetate). In some embodiments, the contrast agent is pH sensitive. For example, 1,4,7,10 tetraazacyclododecane-1,4,7,10-tetraacetic acid (DOTA) may be used for pH sensing. This molecule contains a p-nitrophenol on a twelve-member ring. Under basic conditions, only one water molecule is involved in the coordination, while under acidic conditions, two water molecules will coordinate to Gd. The contrast agent may be an iron oxide, iron platinum, or manganese contrast agent. The contrast agent may be protein contrast agent. The contrast agent should be capable of providing appropriate response to whatever MRI resolution is desired and whatever MRI intensity is used. Additional contrast agents may be found in U.S. Pat. No. 6,321,105, and U.S. Patent Publication US 2015/0202330, each of which is incorporated in their entirety.

Imaging agents can include fluorescent molecules, radioisotopes, nucleotide chromophores, chemiluminescent moieties, magnetic particles, bioluminescent moieties, and combinations thereof. In some embodiments, the composition further comprises a fluorescent dye. The fluorescent dye may be a derivative of rhodamine, erythrosine or fluorescein. The fluorescent dye may be a xanthene derivative dye, an azo dye, a biological stain, or a carotenoid. The xanthene derivative dye may be a fluorene dye, a fluorone dye, or a rhodole dye. The fluorene dye may be a pyronine dye or a rhodamine dye. The pyronine dye may be chosen from pyronine Y and pyronine B. The rhodamine dye may be rhodamine B, rhodamine G and rhodamine WT. The fluorone dye may be fluorescein or fluorescein derivatives. The fluorescein derivative may be phloxine B, rose bengal, or merbromine. The fluorescein derivative may be eosin Y, eosin B, or erythrosine B. The azo dye may be methyl violet, neutral red, para red, amaranth, carmoisine, allura red AC, tartrazine, orange G, ponceau 4R, methyl red, or murexide-ammonium purpurate. Exemplary fluorescent dyes include, but not limited to Methylene Blue, rhodamine B, Rose Bengal, 3-hydroxy-2,4,5,7-tetraiodo-6-fluorone, 5,7-diiodo-3-butoxy-6-fluorone, erythrosin B, Eosin B, ethyl erythrosin, Acridine Orange, 6′-acetyl-4,5,6,7-tetrachloro-2′,4′,5′,6′,7′-tetraiodofluorescein (RBAX), fluorone, calcein, carboxyfluorescein, eosin, erythrosine, fluorescein, fluorescein amidite, fluorescein isothiocyanate, indian yellow, merbromin, basic red 1, basic red 8, solvent red 45, rhodamine 6G, rhodamine B, rhodamine 123, sulforhodamine 101, sulforhodamine B, and Texas Red (sulforhodamine 101 acid chloride). In some embodiments, the compositions and methods disclosed herein may include lipid or protein emulsifiers that improve the stability, drug loading, and drug release efficacy of the system.

The compositions disclosed herein may be administered through any mode of administration. In some aspects, the compositions may be administered intracranially or into the cerebrospinal fluid (CSF). In some aspects, the compositions are suitable for parenteral administration. These compositions may be administered, for example, intraperitoneally, intravenously, or intrathecally. In some aspects, the compositions are injected intravenously. In some embodiments, the compositions are injected into the lymphatic system. In some embodiments, the compositions may be administered enterally or parenterally. Compositions may be administered subcutaneously, intravenously, intramuscularly, intranasally, by inhalation, orally, sublingually, by buccal administration, topically, transdermally, or transmucosally. Compositions may be administered by injection. In some embodiments, compositions are administered by subcutaneous injection, orally, intranasally, by inhalation, into the lymphatic system, or intravenously. In certain embodiments, the compositions disclosed herein are administered by subcutaneous injection.

The terms “individual,” “subject,” “host,” and “patient,” to which administration is contemplated, are used interchangeably herein; these terms typically refer to a mammal, including, but not limited to, murines, simians, humans, mammalian farm animals, mammalian sport animals, and mammalian pets, but can also include commercially relevant birds such as chickens, ducks, geese, quail, and/or turkeys. A mammalian subject may be human or other primate (e.g., cynomolgus monkey, rhesus monkey), or commercially relevant mammals such as cattle, pigs, horses, sheep, goats, cats, and/or dogs. The subject can be a male or female of any age group, e.g., a pediatric subject (e.g., infant, child, adolescent) or adult subject (e.g., young adult, middle-aged adult or senior adult). In some embodiments, the subject may be murine, rodent, lagomorph, feline, canine, porcine, ovine, bovine, equine, or primate. In some embodiments, the subject is a mammal. In some embodiments, the subject is a human. In some embodiments, the subject may be female. In some embodiments, the subject may be male. In some embodiments, the subject may be an infant, child, adolescent or adult.

In some embodiments, disclosed herein is a method of treating or ameliorating one or more symptoms in a model organism that models a neurological disease or disorder selected from Alzheimer's Disease, epilepsy, tremors, seizures, CNS cancers and tumors (gliomas, glioblastoma multiforme (GBM), diffuse instrinsic pontine glioma (DIPG)), pain (including neuropathic pain), and psychiatric diseases (e.g., PTSD, anxiety disorder, depression, bipolar disease, suicidality), wherein the polymeric perfluorocarbon nanoemulsion composition is administered intravenously or into the cerebrospinal fluid (CSF) to the subject/model organism and an uncaging ultrasound pulse is delivered to the subject at an intensity sufficient to yield particle activation (e.g., 1.0 MPa, 50 ms/1 Hz×60 seconds (every second for 60 seconds). In some embodiments, the model organism is a rodent. In some embodiments, the model organism is a rat. In some embodiments, the uncaging ultrasound pulse is delivered to the subject at 1.5 MPa, 50 ms/1 Hz×60 seconds (every second for 60 seconds). In some embodiments, the uncaging ultrasound pulse is delivered to the subject at a pressure between 0.8 and 1.8 MPa, and with a burst length of 10-100 ms. It is to be understood that the method disclosed herein is not limited to the choice of sonication protocol or the specific focused ultrasound transducer, especially because the threshold for activation will be a function of the sonication frequency, the choice of perfluorocarbon, and the particle size.

In some animal model subjects, e.g., rat, a higher frequency of ultrasound is used than may be used in humans. In human subjects, a lower frequency must be used to get through the skull. In some embodiments, disclosed herein is a method of treating or ameliorating one or more symptoms in a subject having a neurological disease or disorder selected from Alzheimer's Disease, epilepsy, tremors, seizures, CNS cancers and tumors (gliomas, glioblastoma multiforme (GBM), diffuse instrinsic pontine glioma (DIPG)), pain (including neuropathic pain), and psychiatric diseases (e.g., PTSD, anxiety disorder, depression, bipolar disease, suicidality), wherein the polymeric perfluorocarbon nanoemulsion composition is administered intravenously or into the cerebrospinal fluid (CSF) of the subject and an uncaging ultrasound pulse delivered to the subject is less than or equal to 1 mega Hz. In some embodiments, subject is a human. In some embodiments, the uncaging ultrasound pulse delivered to the subject is between 220 and 650 kHz. In some embodiments, the uncaging ultrasound pulse delivered to the subject is between 220 and 1000 KHz.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

A “therapeutically effective amount” or “efficacious amount” means the amount of a compound that, when administered to a mammal or other subject for treating a disease, is sufficient to effect such treatment for the disease. The “therapeutically effective amount” will vary depending on the compound, the disease and its severity and the age, weight, etc., of the subject to be treated.

The term “uncaging” refers to the process of inducing oscillations and/or expansion of the core of the nanoparticles, which allows the hydrophobic compound to be released from the nanoparticles.

The term “unit dosage form,” as used herein, refers to physically discrete units suitable as unitary dosages for human and animal subjects, each unit containing a predetermined quantity of compounds/therapeutic agents of the present disclosure calculated in an amount sufficient to produce the desired effect in association with a pharmaceutically acceptable diluent, carrier or vehicle.

As used herein, the phrase “pharmaceutically acceptable carrier” refers to a carrier medium that does not interfere with the effectiveness of the biological activity of the active ingredient. Such a carrier medium is essentially chemically inert and nontoxic.

As used herein, the phrase “pharmaceutically acceptable” means approved by a regulatory agency of the Federal government or a state government, or listed in the U.S. Pharmacopeia or other generally recognized pharmacopeia for use in animals, and more particularly for use in humans.

As used herein, the term “carrier” refers to a diluent, adjuvant, excipient, or vehicle with which the therapeutic is administered. Such carriers can be sterile liquids, such as saline solutions in water, or oils, including those of petroleum, animal, vegetable or synthetic origin, such as peanut oil, soybean oil, mineral oil, sesame oil and the like. A saline solution is a preferred carrier when the pharmaceutical composition is administered intravenously or into the cerebrospinal fluid (CSF). Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The carrier, if desired, can also contain minor amounts of wetting or emulsifying agents, or pH buffering agents. These pharmaceutical compositions can take the form of solutions, suspensions, emulsion, tablets, pills, capsules, powders, sustained-release formulations and the like. The composition can be formulated as a suppository, with traditional binders and carriers such as triglycerides. Examples of suitable pharmaceutical carriers are described in Remington's Pharmaceutical Sciences by E. W. Martin. Examples of suitable pharmaceutical carriers are a variety of cationic polyamines and lipids, including, but not limited to N-(1(2,3-dioleyloxy)propyl)-N,N,N-trimethylammonium chloride (DOTMA) and diolesylphosphotidylethanolamine (DOPE). Liposomes are suitable carriers for gene therapy uses of the present disclosure. Such pharmaceutical compositions should contain a therapeutically effective amount of the compound, together with a suitable amount of carrier so as to provide the form for proper administration to the subject. The formulation should suit the mode of administration.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “nucleic acid” and “polynucleotide” are used interchangeably herein, and refer to a polymeric form of nucleotides of any length, either deoxyribonucleotides or ribonucleotides, or analogs thereof. Non-limiting examples of nucleic acids and polynucleotides include linear and circular nucleic acids, messenger RNA (mRNA), cDNA, recombinant polynucleotides, vectors, probes, primers, single-, double-, or multi-stranded DNA or RNA, genomic DNA, DNA-RNA hybrids, chemically or biochemically modified, non-natural, or derivatized nucleotide bases, oligonucleotides containing modified or non-natural nucleotide bases (e.g., locked-nucleic acids (LNA) oligonucleotides), and interfering RNAs.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi(dot)n|m(dot)nih(dot)gov/BLAST. See, e.g., Altschul et al. (1990), J. Mol. Biol. 215:403-10. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

The terms “double stranded RNA,” “dsRNA,” “partial-length dsRNA,” “full-length dsRNA,” “synthetic dsRNA,” “in vitro produced dsRNA,” “in vivo produced dsRNA,” “bacterially produced dsRNA,” “isolated dsRNA,” and “purified dsRNA” as used herein refer to nucleic acid molecules capable of being processed to produce a smaller nucleic acid, e.g., a short interfering RNA (siRNA), capable of inhibiting or down regulating gene expression, for example by mediating RNA interference “RNAi” or gene silencing in a sequence-specific manner. Design of a dsRNA or a construct comprising a dsRNA targeted to a gene of interest is routine in the art, see e.g., Timmons et al. (2001) Gene, 263:103-112; Newmark et al. (2003) Proc Natl Acad Sci USA, 100 Supp 1:11861-5; Reddien et al. (2005) Developmental Cell, 8:635-649; Chuang & Meyerowitz (2000) Proc Natl Acad Sci USA, 97:4985-90; Piccin et al. (2001) Nucleic Acid Res, 29:E55-5; Kondo et al. (2006) Genes Genet Syst, 81:129-34; and Lu et al. (2009) FEBS J, 276:3110-23; the disclosures of which are incorporated herein by reference.

The terms “short interfering RNA”, “siRNA”, and “short interfering nucleic acid” are used interchangeably may refer to short hairpin RNA (shRNA), short interfering oligonucleotide, short interfering nucleic acid, short interfering modified oligonucleotide, chemically-modified siRNA, post-transcriptional gene silencing RNA (ptgsRNA), and other short oligonucleotides useful in mediating an RNAi response. In some instances siRNA may be encoded from DNA comprising a siRNA sequence in vitro or in vivo as described herein. When a particular siRNA is described herein, it will be clear to the ordinary skilled artisan as to where and when a different but equivalently effective interfering nucleic acid may be substituted, e.g., the substation of a short interfering oligonucleotide for a described shRNA and the like.

“Complementary,” as used herein, refers to the capacity for precise pairing between two nucleotides of a polynucleotide (e.g., an antisense polynucleotide) and its corresponding target polynucleotide. For example, if a nucleotide at a particular position of a polynucleotide is capable of hydrogen bonding with a nucleotide at a particular position of a target nucleic acid, then the position of hydrogen bonding between the polynucleotide and the target polynucleotide is considered to be a complementary position. The polynucleotide and the target polynucleotide are complementary to each other when a sufficient number of complementary positions in each molecule are occupied by nucleotides that can hydrogen bond with each other. Thus, “specifically hybridizable” and “complementary” are terms which are used to indicate a sufficient degree of precise pairing or complementarity over a sufficient number of nucleotides such that stable and specific binding occurs between the polynucleotide and a target polynucleotide.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. As such, an antisense polynucleotide which is 18 nucleotides in length having 4 (four) noncomplementary nucleotides which are flanked by two regions of complete complementarity with the target nucleic acid would have 77.8% overall complementarity with the target nucleic acid. Percent complementarity of an oligomeric compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

The patents, patent applications and publications discussed herein are provided solely for their disclosure prior to the filing date of the present application, and are incorporated by reference herein in their entirety. Nothing disclosed herein is to be construed as an admission that the present disclosure is not entitled to antedate such publication by virtue of prior invention. Further, the dates of publication provided may be different from the actual publication dates which may need to be independently confirmed.

### EXAMPLES

The following examples are set forth to provide those of ordinary skill in the art with a complete disclosure and description of how to make and use the present compositions and methods, and are not intended to limit the scope of what the inventors regard as their invention nor are the examples intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. Standard abbreviations may be used, (e.g., “bp” refers to base pair(s); “kb” refers to kilobase(s); “ml” refers to milliliter(s); “s” or “sec” refers to second(s); “min” refers to minute(s); “h” or “hr” refers to hour(s); “aa” refers to amino acid(s); “nt” refers to nucleotide(s); “i.v.” or “IV” refers to intravascular(ly); and the like.

### Materials and Methods

Equipment used herein: Bransonic Series Model M1800H (40 kHz), Liposofast™ L-50 membrane extruder; Malvern Zetasizer Nano ZS90, TECAN Infinite spectrophotometer, Thermo Scientific Sorvall RC6+ centrifuge.

Chemicals: Di-block copolymers are made up of a hydrophilic block of polyethylene glycol (PEG; mol. wt. 2 kDa) and a hydrophobic block of one of: poly(lactic-co-glycolic acid) (PLGA), poly(L-lactic acid)(PLLA), or poly(ε-caprolactone) (PCL). Two molecular weights of hydrophobic block chains were used: 2 kDa and 5 kDa. The example of nomenclatures for di-block copolymer is polyethylene glycol 2 kDa-poly(lactic-co-glycolic acid)=PEG (2 kDa)−PLGA (5 kDa). All diblock copolymers were purchased from Akina (West Lafayette, IN, USA). Propofol, nicardipine hydrochloride, verapamil hydrochloride, sodium sulfate and sodium hydroxide were purchased from Alfa Aesar (Haverhill, MA, USA). Doxorubicin hydrochloride was purchased from LC laboratories (Woburn, MA, USA). Cisplatin and dexmedetomidine were purchased from Sigma-Aldrich (St Louis, MO, USA). Ketamine hydrochloride injectable solution is a controlled substance and was purchased via Stanford University Environmental Health & Safety. Tetrahydrofuran (THF), methanol, ethyl acetate, chloroform, and hexane were obtained from Sigma-Aldrich (St Louis, MO, USA). n-Perfluoropentane (PFP) was purchased from FluoroMed (Round Rock, TX, USA). A hydrophobic IRDye® 800RS infrared dye was purchased from LICOR Biotechnology (Lincoln, NE, USA).

Removal of Hydrochloride from Drug Molecules: The base form of ketamine, doxorubicin, nicardipine, and verapamil was prepared by removing hydrochloride from purchased chemicals for further encapsulation in the nanoemulsions. The drug molecule was dissolved in a proper solvent (doxorubicin hydrochloride: 2 mg/ml in DI water; ketamine hydrochloride: 2 mg/ml in saline; nicardipine hydrochloride: 2 mg/ml in 2:1 DI water:methanol (v:v); verapamil hydrochloride: 2 mg/ml in DI water). 3 N NaOH was added to neutralize the hydrochloride. A two-fold volume of chloroform was used to extract the drug base molecule from the aqueous phase three times. The combined chloroform phase was dried over anhydrous sodium sulfate. After complete evaporation of chloroform, the drug was sealed in a glass vial and stored at −20° C. for future use.

### Method of Producing Propofol-Loaded Nanoparticles:

150 mg of a block polymer was weighed into a 40 ml glass beaker, and 10 ml tetrahydrofuran (THF) was added to the beaker. This mixture was magnetically stirred at room temperature until completely dissolved. Once the mixture was dissolved, 15 μl propofol was added and the solution was stirred for another 5 minutes. 10 ml PBS was then added and the solution was stirred overnight to fully evaporate the THF.

After the overnight incubation, 0.3 ml PFP was added and the solution was placed on ice for 15 minutes. Next, the solution was vigorously pipetted up and down 10-15 times (to mix the PFP that has settled to the bottom with a 1 ml micropipette, and then sonicated for 3-5 minutes in a bath sonicator (40 kHz; Model: Bransonic M1800H) filled with iced water.

The nanoparticle solution was then transferred into a 50 ml Falcon™ tube and centrifuged at 4° C. and 2000 g for 10 min. The supernatant was removed and the nanoparticles were re-suspended in 10 ml cold PBS with a pipette and again centrifuged at 4° C. and 2000 g for 10 min. This resuspension and centrifugation was repeated two more times (four times in total). At the end of this procedure, the final resuspension in a 10 ml cold PBS yields approximately 0.5-0.6 g of nanoparticles. The nanoparticle solution was then filtered twice using a Liposofast™ LF-50 membrane extruder equipped with compressed nitrogen and loaded with polycarbonate membrane of 0.6 μm pores.

Pharmacokinetics and Biodistribution of Drug-Loaded Nanoemulsions comprising nanoparticles: All animal experiments were carried out in accordance with the Stanford IACUC. Long-Evans rats with body weight 180-200 g (Charles River Laboratories, Wilmington, MA, USA) were used in all in vivo studies. Drug-loaded PFP/PEG (2 kDa)-PLGA (5 kDa) nanoemulsions were doped with a hydrophobic near infrared fluorescent dye, IR800 (LI-COR, Lincoln, NE), during nanoemulsion production. Propofol, nicardipine, and doxorubicin-loaded nanoemulsions were used to test in vivo blood-pool particle kinetics and systemic biodistribution.

To produce dye-doped nanoemulsions, 1 mg IR800 dye was added to the drug and polymer THF solution, and the rest of the nanoemulsion production protocol was unchanged. For the experiments, a nanoemulsion bolus (equivalent to 1 mg/kg of drug) was administered intravenously via a 24 g×¾″ catheter paced in the rat tail vein in a total volume of ˜0.4-0.5 ml (N=3). Blood samples were collected via either left or right submandibular vein at 2 min 10 min, 20 min, 40 min, 2 h and 4 h, alternating sides for each sampling. The blood was split into two volumes. Whole blood sample fluorescence was assessed using a Lago (Spectral Instruments Imaging; Tucson, AZ, USA) imaging system (excitation/emission=770/810 nm) and quantification was completed using regions of interest (ROIs) of the same size across samples, drawn to be within the capillary tube. The second volume of each sample was microcentrifuged for a total of 10 min at 10,000 g at 4° C. The plasma fraction from these samples was then collected and their fluorescence was quantified similar to that of whole-blood samples. The nanoemulsion concentration in the whole blood and plasma were fitted with a two-compartment kinetic model. The clearance kinetics of dye-doped propofol-loaded nanoemulsions administered as a bolus (equivalent to 1 mg/kg of propofol) followed by an immediate infusion (equivalent to 1.5 mg/kg/hr of propofol) was also quantified. Blood was collected and quantified as described above.

For systemic biodistribution, the same dye-doped nanoemulsions (propofol, nicardipine, or doxorubicin-loaded) were administered intravenously as a bolus to Long-Evans rats (N=3). The rats were sacrificed at 24 h post administration to harvest major organs: heart, liver, lungs, kidneys, spleen, and brain. These organs were imaged for IR800 fluorescence (excitation/emission=770/810 nm) using a Lago imaging system and quantified using regions of interest (ROI) of the same size, drawn to be within the image of each organ. The distribution of the nanoemulsion among the organs was calculated by dividing the ROI fluorescence of each tissue by the sum of ROI fluorescence values of all organs.

### EEG/VEP Measurement

Electrode Implantation: The skin on the rat head (body weight 180-200 g) was carefully removed with clippers. A 9-mm skin incision on the head was made and 1-mm burr holes were drilled into the skull for two-electrode implantation. A stainless-steel skull screw (J.I. Morris, Southbridge, MA, USA) was implanted through the skull close to the visual cortex (6 mm posterior to bregma and 1 mm lateral to midline) as the signal electrodes A reference screw electrode was placed 2 mm anterior to bregma and 2 mm lateral to midline. Dental cement (BASi, West Lafayette, IN, USA) was used to fix the screws. The skin incision was closed and 10 days were allowed for the animals to recover from the surgery before electroencephalography (EEG) recording.

EEG Recording and LED Stimulus Setup: EEG recording was performed with an 8 Channel Cyton Biosensing Board (OpenBCI, Brooklyn, NY, USA) with a custom firmware allowing for a sampling rate of 500 Hz along with recording of stimulus timings. To prevent aliasing, samples were recorded at 16 kHz with digital filtering before resampling at 500 Hz. The OpenBCI board was also modified to interface with a laptop via a USB breakout board (Adafruit, NY, USA) and USB isolator (Adafruit, NY, USA). For EMI shielding, the box was placed in a Faraday Cage consisting of a cardboard box with aluminum foil and copper tape. Stimulus was provided by a Mini-Ganzfeld Stimulator consisting of a 3D-printed cone with three green LEDs (Linrose B4304H5-10, Plainview, NY, USA) embedded, shielded with black electrical tape and copper mesh shielding. A Raspberry Pi 2 Model B (RS Components Ltd., Corby, Northants, UK) was used to coordinate stimulus delivery, connected to a breadboard (Twin Industries, San Ramon, CA) and a MOSFET (NTE, Bloomfield, NJ) to gate LED stimulus.

Combined FUS-EEG Setup: At least 10 days after electrode implantation, animals were anesthetized with ketamine/xylazine and were placed in a plastic stereotactic frame (Image Guided Therapy, Pessac, France) coupled to the FUS system, and immobilized with ear bars and a bite bar. Any remaining dorsal scalp fur in the sonication trajectory was removed by clipping and applying a chemical depilatory (Nair, amazon.com). A hair dryer was used for 20-30 s to remove moisture from around the electrodes. The signal and reference electrodes were coupled to the corresponding skull screw electrodes and the custom-made EEG system. A needle was inserted under the skin of the neck as the ground electrode. A digital multimeter was used to ensure that the electrode impedances were below 5Ω. A monocular visual stimulus (Linrose B4304H5-10; 10 ms flashes presented at 1 Hz) was applied contralateral to the sonicated hemisphere and the ipsilateral eye was covered with a plastic cone. A thin (<1 mm) ultrasound pad (Aquaflex®, Parker Laboratories, Inc., Fairfield, NJ, USA) was used to couple the FUS transducer membrane and the skin of the head. To account for skull attenuation, a 30% pressure insertion loss was assumed for this size and age of rats2. Prior to recording, animals were kept in a darkened room and allowed to adapt to darkness for at least 5 minutes.

Visual Evoked Potential (VEP) Recording: A total of 3 animals were assigned for EEG recording. To ensure an adequate anesthesia plane was achieved to yield the appropriate signal-to-noise ratio for the experiment, the VEP amplitude was monitored and the experiment proceeded only if the VEP N1P1 amplitude measured at least 60 μV. Once this condition was achieved, 6 min VEP traces were acquired, with either focused ultrasound or nanoemulsion intravenous administration commencing at 3 min after the VEP recording started. At least 10 min passed between nanoemulsion administration and the next FUS application.

EEG Data Analysis: Data analysis was performed in Python. Raw EEG traces were digitally filtered with a 4th order bandpass Butterworth filter with cutoff frequencies of 1-100 Hz. Notch filtering for 60 Hz noise and its higher harmonics consisted of 2nd order digital Chebyshev filters with cutoff frequencies of 58-62 Hz, 118-122 Hz, 178-182 Hz, and 238-242 Hz. VEP traces were computed by averaging over all presented VEP stimuli over a 60 second period with a Gaussian kernel with a standard deviation of 20 seconds. N1P1 amplitude for averaged VEP traces was quantified by finding the first local minimum 40 ms after stimulus onset and finding the next local maximum, and taking the difference. Traces consisting of N1P1 amplitudes that had swings between adjacent presentations of more than 30 μV in either direction were excluded because they were indicative of VEP traces that were too unstable to quantify.

Flow Channel Phantom Experiment: B-mode and Doppler images were acquired with a Siemens Acuson S2000 scanner (Siemens Healthcare Diagnostics, Tarrytown, NY, USA) and a Siemens 4C1 transducer (Siemens Healthcare Diagnostics, Tarrytown, NY, USA) using a transmit frequency of 3 MHz for B-mode and 2.5 MHz for power Doppler. The phantom experiments were performed on an ATS Laboratories model 523A Doppler phantom (ATS Laboratories, Bridgeport, CT, USA) with a 4 mm vessel phantom. Heparinized bovine whole blood (Innovative Research, Novi, MI USA) was used as a control and administered through the phantom at a flow rate of 58 mL/min. The flow rate was determined based on the average intracranial blood flow rate in humans. The nicardipine-loaded nanoemulsions were prepared in bovine whole blood and used at 0.265 mg/ml nicardipine concentration for the indicated experiments.

### Example 1

### Production of Drug-Loaded Polymeric Perfluoropentane Nanoemulsions

The production of polymeric perfluoropentane (PFP) nanoemulsions was similar for all the tested drugs and amphiphilic di-block copolymers. Briefly, 150 mg of di-block copolymer and 15 mg of drug were weighed into a 20 ml glass beaker and 10 ml THF was added to dissolve the polymer and drug. Then, 10 ml phosphate buffer saline (PBS) was added dropwise to the organic solution over 5 min. The THF was fully evaporated by placing the mixture overnight in atmosphere and then in vacuum for 1 h. This produced drug-loaded polymeric micelles in saline suspension. Then, 300 μl cold PFP was added to the suspension, followed by 5 min sonication in a 40 KHz Bransonic M1800H bath sonicator (Thermo Scientific; Waltham, MA, USA) which was pre-filled with iced water. The solution was centrifugated at 4° C. at 2000 g for 10 min. The supernatant was decanted and the resulting pellet was resuspended in cold PBS. Centrifugation-resuspension was repeated two more times to remove and dilute residual free drug, polymer, and PFP-free micelles. Finally, the nanoemulsion suspension was extruded twice using an Avestin Liposofast LF-50 extruder (Ottawa, ON, Canada) equipped with compressed nitrogen (40 psi) and loaded with a polycarbonate membrane of 0.6 μm pores. The extruded nanoemulsion suspension was either used fresh or mixed with glycerin (2.25%, w/v) and frozen immediately and stored at −80° C. until it was thawed for use. Prior to finalizing the production protocol, the volume percentage of PFP to nanoemulsion solution (between 0.2, 0.5, 1.0, 3.0 and 6.0%) was varied to find an appropriate set of physiochemical characteristics for ultrasonic drug uncaging. The preliminary screening was performed with the di-block copolymer PEG (2 kDa)-PCL (2 kDa) using the same procedure.

### Example 2

Nanoparticle Characterization: To determine the hydrodynamic diameter and polydispersity index (PDI) of propofol-loaded PFP/PEG2k-PCL2k nanoparticles:

Briefly, 20 μl of the nanoparticle solution from Example 1 was transferred to 1 ml 4° C. PBS. The sample solution was thoroughly vortexed for 5 sec. The hydrodynamic diameter and polydispersity index were measured with Malvern Zetasizer Nano ZS90. A single peak at ˜390 to 450 nm (measured by intensity) was obtained.

Propofol loading in PFP/PEG2k-PCL2k nanoparticles: A 100 μl nanoparticle solution was thoroughly mixed with 900 μl methanol. The fluorescence of propofol was measured for quantifying the loading of propofol in nanoparticles (Excitation=276 nm/Emission=302 nm). The propofol loading was calculated based on an established calibration curve in the same solution.

TABLE 1
Physicochemical properties of various
propofol-loaded polymer nanoparticles

| | | Polydispersity | Drug loading |
|---|---|---|---|
| Polymer | Size (nm) | index | %, wt |
| PCL2k | 424 ± 14.7 | 0.106 ± 0.028 | 0.47 ± 0.03 |
| PCL5k | 450 ± 28.2 | 0.163 ± 0.051 | 0.70 ± 0.04 |
| PLGA2k | 395 ± 15.4 | 0.122 ± 0.038 | 0.51 ± 0.03 |
| PLGA5k | 436.4 ± 24.3 | 0.071 ± 0.052 | 0.75 ± 0.02 |
| PLLA2k | 464.2 ± 8.8 | 0.152 ± 0.028 | 0.52 ± 0.01 |
| PLLA5k | 735.8 ± 49.4 | 0.448 ± 0.049 | 2.03 ± 0.01 |

From these results, it was observed that the average size of the nanoparticles made from each of the various polymers was below 500 nm except the nanoparticles made from PEG2k-PLLA5k. It was also noted that the propofol loading increases with as the length of hydrophobic polymer block (i.e. PCL, PLLA, and PLGA) increases.

TABLE 2
Physicochemical properties of PFP/PCL2K-PEG2K
nanoparticles loaded with various drugs

| | | | Polydispersity | Drug loading |
|---|---|---|---|---|
| Polymer | LogP | Size (nm) | index | %, wt |
| Propofol | 3.79 | 424 ± 14.7 | 0.106 ± 0.028 | 0.47 ± 0.03 |
| Nicardipine | 3.82 | 431.8 ± 26.3 | 0.170 ± 0.06 | 0.66 ± 0.05 |
| Dexmedetomidine | 2.8 | 444.4 ± 4.2 | 0.173 ± 0.044 | 0.37 ± 0.02 |
| Ketamine | 2.18 | 422.5 ± 21.3 | 0.151 ± 0.03 | 0.61 ± 0.04 |
| Verapamil | 3.79 | 427.2 ± 13.1 | 0.093 ± 0.038 | 0.72 ± 0.03 |
| Doxorubicin | 1.27 | 404.7 ± 11.2 | 0.118 ± 0.05 | 0.30 ± 0.04 |
| Cisplatin | 0.04 | 409.5 ± 16.3 | 0.107 ± 0.03 | 0.06 ± 0.01 |

From these results, it was observed that drug loading generally increases as the Log P of the drug increases. (Log P is a measure of drug hydrophobicity). Drug loading increased with hydrophobicity.

### Example 3

In Vitro Release of Propofol from Propofol-Loaded Nanoparticles Made From Various Polymers:

A PCR tube filled with 100 μl nanoparticle solution and 200 μl hexane on the top was situated on a custom-designed focused ultrasound (FUS) transducer which was immersed in water bath. The particles were sonicated with the transducer (1.5 MHz center frequency) at 0.5 Hz burst frequency for 2 min (60 bursts) at a variety of in situ pressure (MPa). Following FUS, 100 μl of the hexane phase was removed without disturbing the aqueous layer, and this was diluted with 100 μl hexane. The propofol concentration was quantified by UV fluorescence at excitation=276 nm/emission=304 nm and compared to a standard curve of propofol in hexane.

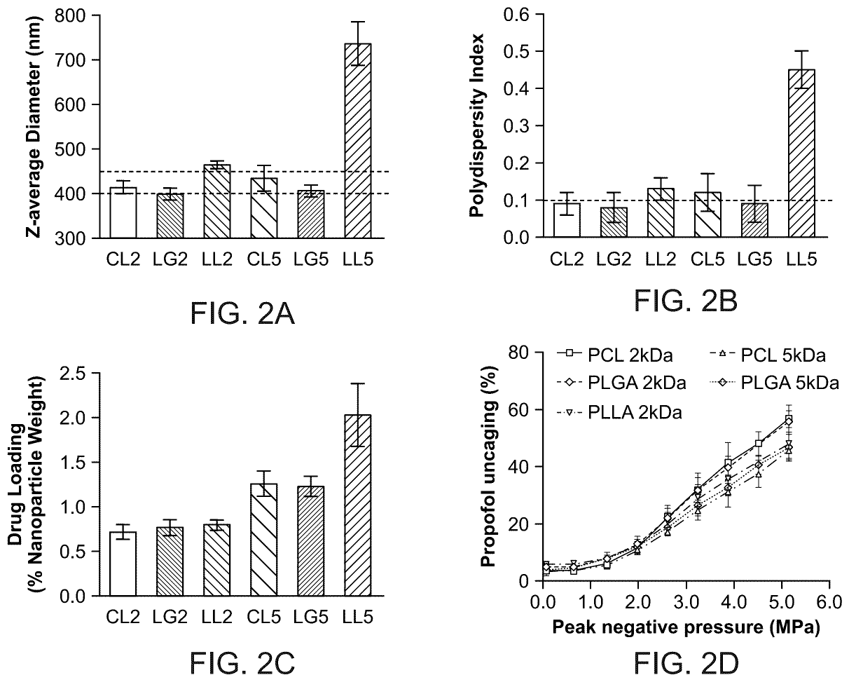

Referring to FIG. 2d, the in vitro release characteristics were assessed for the nanoparticles made with various hydrophobic block co-polymers and PEG2k as the hydrophilic block. From these results, it was observed that the pressure threshold at which the propofol was released was 1.3 MPa for 1.5 MHz sonication for all polymers except PLLA2k (FIG. 1). In some embodiments, the pressure threshold of release was 0.8 MPa for 650 KHz sonication. Furthermore, it was noted that the drug release increases as the length of hydrophobic polymer block decreases. This may be explained by the different surfactant properties of the polymers.

### Example 4

In Vitro Stability of Propofol-Loaded PFP/PEG2k-PLGA5k and PFP/PEG2k-PCL2k Nanoparticles:

The in vitro stability of propofol-loaded PFP/PEG2k-PLGA5k and PFP/PEG2k-PCL2k nanoparticles was tested at temperatures of 4° C. and 37° C., the former being a likely storage temperature and the latter representing physiologically relevant human body temperature. PFP/PCL2k-PEG2k nanoparticles: The polymeric perfluorocarbon nanoemulsion comprising nanoparticles comprising PFP/PCL2k-PEG2k made by the methods disclosed herein were found to have desirable physicochemical properties (i.e., stable hydrodynamic diameter and PDI in at least the first 0-1.5 hours at 4° C. The size and polydispersity increase over time at both at 4° C. and at 37° C., and the nanoparticles are not detected after 24 hours at 37° C., likely due to the evaporation of the liquid core of the nanoparticles. PFP/PLGA5k-PEG2k nanoparticles: The nanoparticles comprising PFP/PLGA5k-PEG2k made by the methods disclosed herein were found to have desirable physicochemical properties (i.e., stable hydrodynamic diameter and PDI in at least the first 0-3 hours at 4° C. At 4° C., both size and polydispersity increase slightly after 3-4 hrs of storage, and at 37° C. the increases in size and PDI is significant.

Physicochemical Characterization of Drug-loaded Polymeric c Perfluoropentane Nanoemulsions Dynamic Light Scattering (DLS): The Z-average diameter, polydispersity index (PDI) and zeta potential of the drug-loaded nanoemulsions were measured with a Malvern Zetasizer Nano ZS90 (Malvern, United Kingdom). A 10 μl nanoemulsion solution was thoroughly mixed with 990 μl cold PBS. DLS parameters were: materials=perfluoropentane; Refractive Index (RI)=1.330; absorption=0.1; dispersant=ICN PBS tablets; viscosity=0.8882 cP at 25° C.; Mark-Houwink parameters; equilibration time=60 s; disposable cuvettes=ZEN0118; measurement angle=90 degree; measurement duration=automatic; number of measurements=5; positioning method=seek optimum position; analysis model=general purpose (normal resolution). To measure the zeta potential, 10 μl nanoemulsion solution was mixed with 990 μl deionized water. Then 900 μl of this solution was transferred to a disposable capillary cell. Measurement parameters were: cell type=DTS1070, dispersant=water; viscosity=0.8872 cP at 25° C.; dielectric constant=78.54; F(Ka) selection model=Smoluchowski; F(Ka) value=1.5; measurement duration=automatic; measurement runs between 10 and 100; number of measures=3 with no delay between measurements.

### Example 5

cGMP Compatible and Scalable Production:

For in vivo and clinical applications, the nanoparticle production methods were first adapted for cGMP compatibility and scalability (FIG. 1a). Given prior theory and evidence that similar particles increase their diameter up to a maximum of 5-6 fold during uncaging, a median Z-average diameter of 400-450 nm was targeted, calculated so that during uncaging the nanoparticles would be at most half a capillary diameter. A median polydispersity index (PDI) of <0.1 was also targeted. A typical example of DLS spectrum of perfluoropentane polymeric nanoemulsions was found to have a Z-average diameter was 405.3±22.7 nm, a polydispersity index of 0.061±0.033, mean±S.D. for N=3. The PFP content in the reaction was noted to significantly affect the particle size, drug loading, and monodispersity, and it was empirically determined that a 2 μL:1 mg ratio of PFP to polymer most reliably met the target size and PDI. To generate the nanoparticles, the emulsifying polymer and drug were dissolved in tetrahydrofuran (THF), and then sterile phosphate-buffered saline (PBS) was added. The THF was evaporated to completion, leaving drug-loaded polymeric micelles in saline suspension. Then, PFP was added and the mixture was sonicated in a bath sonicator until the PFP was visibly completely emulsified. Following three cycles of centrifugation and resuspension to remove free drug, polymer, and micelles, the nanoparticles were filtered twice through a membrane extruder to produce the final product. Notably, the shift from immersion sonication, as used previously, to bath sonication and membrane extrusion substantially improved the free drug fraction (FIG. 1b). Dynamic light scattering confirmed that the current methods produced monodisperse peaks of nanoscale material.

FIG. 1 shows nanoparticle production for enhanced stability and efficacy in vitro. FIG. 1a: Schematic of nanoparticle production and ultrasonic drug uncaging. FIG. 1b: Free propofol content is improved with the current optimized protocol versus the prior. FIGS. 1c-1e: Glycerin serves as a cryoprotectant to improve nanoparticle stability through frozen storage and thawing. FIG. 1f: Experimental schematic to assay ultrasonic drug uncaging efficacy in vitro. FIG. 1g: Intact ultrasonic drug uncaging efficacy in vitro (650 KHz sonication, 60×50 ms pulses at 1 Hz pulse repetition frequency) for frozen & thawed nanoparticles compared to fresh. Mean+/−S.D. are presented for groups of N=3. In early formulations, the particle size, free drug fraction, and polydispersity all increased substantially over the course of hours with incubation on ice or at room temperature, and the particles were too unstable to permit a freeze-thaw cycle. To address this significant practical limitation, cryoprotectants were used to enable frozen storage of the particles. In some embodiments, one or more cryoprotectant(s) are added to the polymeric perfluorocarbon nanoemulsion, such as, for example, glycerin or sucrose. In some embodiments, glycerin or sucrose is used at about 1%, about 1.25%, about 1.5%, about 1.75%, about 2%, about 2.25%, about 2.5%, about 2.75%, or about 3% volume to weight in the polymeric perfluorocarbon nanoemulsion composition. While the addition of 2.25% v/w glycerin to the particles had no substantial effect on the physicochemical characteristics and drug loading of the particles (FIG. 1c), it allowed for improved particle stability in the post-thaw time period (FIG. 1d) and permitted long-term frozen storage of the particles (FIG. 1e) with stability across multiple freeze-thaw cycles. This formulation also showed low batch-to-batch variability with no change of physicochemical characteristics across varied particle concentrations. In this protocol, there was a slow increase of the free drug fraction during room temperature incubation, rising from ˜4% of the initial drug load immediately post-thaw to ˜8% at 3 hours (FIG. 1d). Therefore, the particles are generally used within 3 hours of thawing.

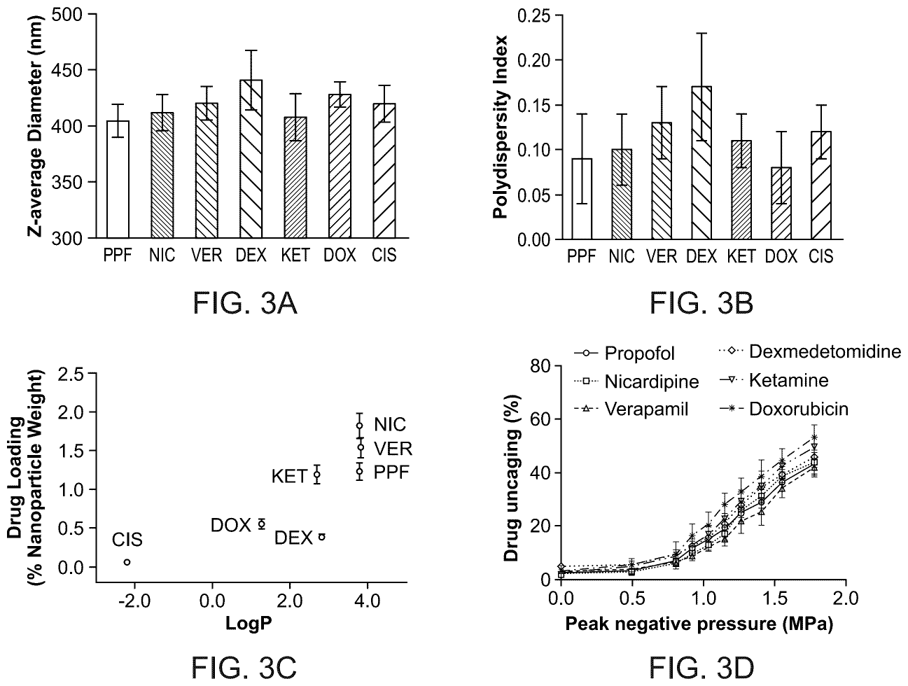

To determine the efficacy of the particles for ultrasonic drug uncaging in vitro, the particles were loaded into thin-walled plastic (PCR) tubes and then added a layer of organic solvent on top that was immiscible with and of lower density than water (FIG. 1f). Following focused sonication of the aqueous nanoparticle suspension, the organic layer was collected, and the UV fluorescence of this fraction was measured to indicate the amount of drug release. Indeed, there was robust FUS-induced drug release seen with a dose-response relationship with the applied in situ peak pressure, and no change of this efficacy between fresh and frozen/thawed nanoparticles, irrespective of the length of time that the particles were frozen (FIG. 1g). With 650 kHz sonication, a drug release sonication pressure threshold of 0.8 MPa was estimated (FIGS. 1g, 3d), with an estimated threshold of 1.2 MPa at 1.5 MHZ (FIG. 2d). Drug release increased generally with sonication burst length, with saturation of the effect near 50 ms.

### Example 6

Pharmacokinetics and Biodistribution of PFP/PEG2k-PCL2k Nanoparticles Doped with a Hydrophobic Dye:

Propofol-loaded nanoparticles doped with an infrared fluorescent dye IR800 (LICOR Biosciences; Lincoln, NE) with maximum excitation at 770 nm and emission at 794 nm were prepared under sterile conditions, as described in the methods herein. The nanoparticles were administered intravenously via a 24×3/4 g tail vein catheter to rats (N=3) in a total volume of 1 ml. Timed submandibular blood collection were performed at 10 min, 20 min, 40 min, 2 hours, 4 hours, 8 hours and 24 hours. The blood sample was split into two volumes. Whole blood sample fluorescence was assessed using a Lago imaging system and quantification was completed using regions of interest of the same size across samples, drawn to be within the capillary tube. As second volume of each sample was centrifuged in a microcentrifuge for a total of 10 min. The serum fraction from these samples was then collected and its fluorescence was quantified similar to the whole-blood samples. A second volume of each sample was centrifuged in a microcentrifuge for a total of 10 min. The serum fraction from these samples was then collected and the fluorescence was quantified similar to the whole-blood samples. The rats were sacrificed 24 h after i.v. injection to harvest major organs (i.e., heart, liver, lungs, kidneys, spleen and brain). These organs were imaged for fluorescence (Excitation=770 nm/Emission=810 nm). Organ fluorescence was also assessed via the Lago imaging system and quantified using regions of interest of the same size drawn to be within the image of each organ.

To determine the effect of the encapsulating diblock copolymer on drug loading and release efficacy, the hydrophobic block of the polymer was varied between the common polymeric drug delivery materials of polycaprolactone (PCL), poly-L-lactic acid (PLLA), and poly-lactic-co-glycolic acid (PLGA). The molecular weight of these blocks was varied between 2 kDa and 5 kDa. The hydrophilic block of poly-ethylene glycol (PEG; mol. wt. 2 kDa) was kept constant. PLLA particles, particularly with a block molecular weight of 5 kDa, showed increased size and polydispersity, and in many cases developed a precipitate during production (biasing the drug loading estimates), indicating that this polymer was not suitable for these applications (FIG. 2). There was minimal difference between PCL and PLGA in terms of the resultant particle physicochemical characteristics and drug loading. Larger hydrophobic blocks yielded greater drug loading (FIG. 2c), with approximately double the drug loading with 5 kDa hydrophobic block sizes compared to 2 kDa. There was minimal difference among the particles in terms of in vitro ultrasonic drug uncaging efficacy (FIG. 2d), with larger hydrophobic blocks trending towards minimally decreased percent uncaging. Given the substantially improved drug loading relative to this minimally decreased percent uncaging, and the greater reported experience of safety and efficacy in clinical drug delivery applications with PLGA compared to PCL, PEG(2 kDa)-PLGA(5 kDa) was chosen as the emulsifying polymer of the nanoemulsions for subsequent experiments.

FIG. 2 shows various polymer choices for compound-loaded polymeric perfluoropentane nanoemulsions. Diblock copolymers were tested consisting of a hydrophilic block of PEG (2 kDa) and a choice of hydrophobic block among: PCL (2 kDa, CL2 or 5 kDa, CL5), PLGA (2 kDa, LG2 or 5 kDa, LG5), or PLLA (2 kDa, LL2 or 5 kDa, LL5) FIG. 2a: Z-average diameter (dashed lines at the target values of 400-450 nm). FIG. 2b: polydispersity index (dashed lines at target value of 0.1). FIG. 2c: propofol drug loading. FIG. 2d: ultrasonic propofol uncaging in vitro (1.5 MHZ sonication, 60×50 ms pulses at 1 Hz pulse repetition frequency) was quantified. Mean+/−S.D. are presented for groups of N=3. In some embodiments, the in vivo intra-vascular concentration (as measured by fluorescence) of IR800-doped PFP/PCL2k-PEG2k nanoparticles in whole blood decreased over time, with a calculated half-life (t½) of between 20 and 40 minutes.

### Example 7

### In Vitro Assay of Ultrasonic Drug Uncaging

The effect of polymer composition and drug partition coefficient (Log P) on in vitro drug uncaging from nanoemulsions were studied. Propofol was used as a model drug to study the effect of the varying hydrophobic polymer blocks. A 50 μl nanoemulsion suspension (1 mg/ml drug equivalent) was added to a Fisherbrand™ 0.2 ml PCR tube (Fisher Scientific). A 150 μl organic solvent of density less than water was added atop the nanoemulsion suspension. The exact solvent used varied depending on the drug being tested: hexane was used to extract propofol and ketamine; ethyl acetate was used for nicardipine, verapamil, dexmedetomidine, and doxorubicin. The PCR tube was placed in a custom holder and coupled using degassed water to a focused ultrasound (FUS) transducer (Image Guided Therapy, Pessac, France) at room temperature, so that the FUS focus was contained within the nanoemulsion suspension layer (FIG. 1f). The nanoemulsions were sonicated with FUS for 60 s total, with varying peak negative pressure, using cycles of 50 ms ultrasound on and 950 ms off, i.e. pulse repetition frequency of 1 Hz. The center frequency of the transducer was 1.5 MHz or 650 KHz. Following FUS, 100 μl of the organic solution was collected without disturbing the aqueous layer. The amount of the uncaged drug was quantified by measuring its UV or fluorescence and comparing to a standard curve of the drug prepared in varying concentrations in the same organic solvent. PEG (2 kDa)-PLGA (5 kDa) was used to create all nanoemulsions for the analysis of how the drug Log P affects nanoemulsion characteristics. The experimental setup and procedure were otherwise similar.

### A Generalized Platform for Drug Delivery

To realize the promise of this system as a platform for targeted delivery of a wide variety of drugs, and to estimate the drug features that most enable encapsulation into polymeric perfluoropentane nanoemulsions, the drug was varied between seven molecules: two vasoactive agents (calcium channel antagonists verapamil and nicardipine), three anesthetics (propofol, ketamine, and dexmedetomidine), and two chemotherapeutics (doxorubicin and cisplatin). There was minimal difference of the encapsulated drug on the particle physicochemical properties (FIGS. 3a,b). Instead, there was a strong positive relationship noted between the drug Log P (a measure of hydrophobicity) and the drug loading (FIG. 3c), with essentially no loading of the hydrophilic compound cisplatin. Interestingly, while there were minimal differences of in vitro ultrasonic drug uncaging efficacy across the different drugs (FIG. 3d), there was a reverse trend compared to drug loading in that doxorubicin (Log P=1.3) had the greatest drug release versus applied pressure, compared to verapamil or nicardipine (Log P=3.8). These results establish the generalizability of this system for ultrasonic uncaging of hydrophobic drugs.

### Quantification of Drug Loading in Nanoemulsions

a. Propofol and doxorubicin. A 100 μl nanoemulsion solution was thoroughly mixed with 900 μl methanol. The fluorescence of drug was quantified with a Tecan Infinite M1000 microtiter plate reader (San Jose, CA, USA) for propofol at excitation/emission=276/302 nm and doxorubicin at 500/595 nm, respectively. The drug content was calculated with respect to a standard curve of the drug prepared in varying concentrations in the same solvent.
b. Ketamine, nicardipine, verapamil and dexmedetomidine. A 100 μl nanoparticle solution was thoroughly mixed with 900 μl methanol. The UV absorption was measured with a Varian Cary 50 UV-VIS spectrophotometer (Agilent Technologies; Santa Clara, CA, USA) for ketamine at 280 nm, nicardipine at 348 nm, verapamil at 282 nm and dexmedetomidine at 262 nm, respectively. The drug content was calculated with respect to a standard curve of the drug prepared in varying concentrations in the same solvent.
c. Cisplatin. The amount of cisplatin encapsulated in the nanoemulsion was measured according to a previously reported method with minor modifications1. A 100 μl nanoemulsion suspension was added to 1.9 ml pH 6.8 PBS (10 mM) and then mixed up with 1 ml orthophenylenediamine (OPDA) DMF solution (1.4 mg/ml). The mixture was heated at 105° C. for 20 min. The solution was cooled down to room temperature and the UV absorbance at 703 nm was immediately measured with a Varian Cary 50 UV-VIS spectrophotometer. The content of cisplatin was calculated with respect to a standard curve of the drug prepared in varying concentrations in the same solvent.

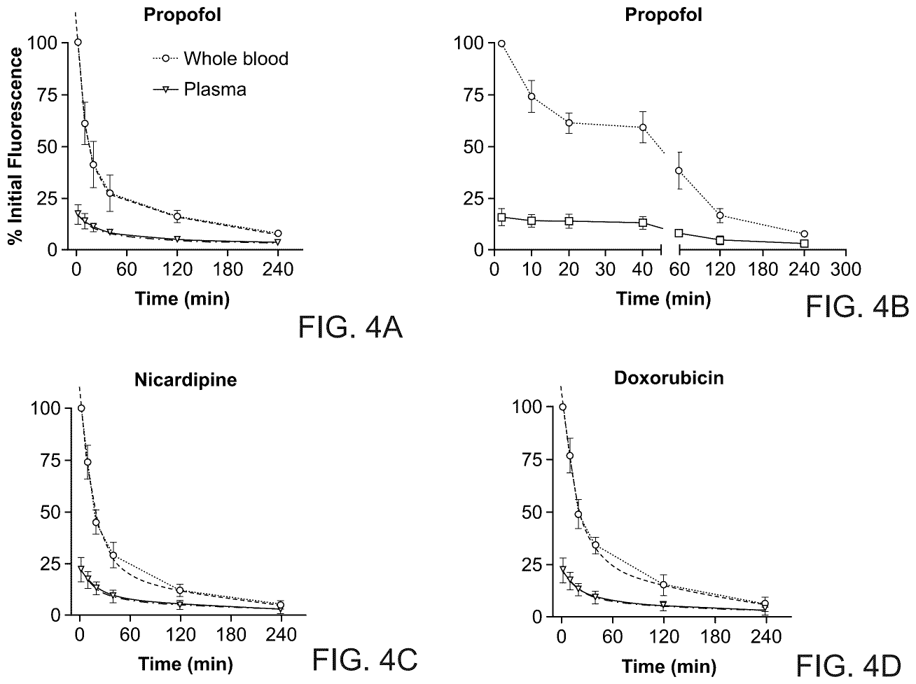

FIG. 4 shows particle clearance kinetics, biodistribution, and biotolerance. Particle kinetics after intravenous administration of 4a, propofol-loaded nanoparticles (bolus of 1 mg/kg encapsulated propofol), 4b, propofol-loaded nanoparticles as an i.v. infusion (bolus of 1 mg/kg+infusion of 1.5 mg/kg/hr encapsulated propofol), 4c, nicardipine-loaded nanoparticles (bolus of 1 mg/kg encapsulated nicardipine), and 4d, doxorubicin-loaded nanoparticles (bolus of 1 mg/kg encapsulated doxorubicin) are shown.

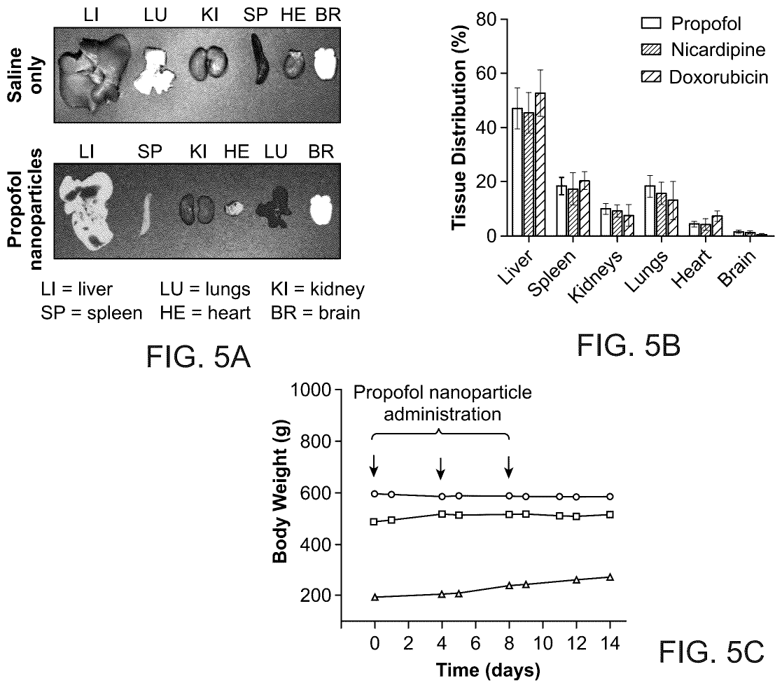

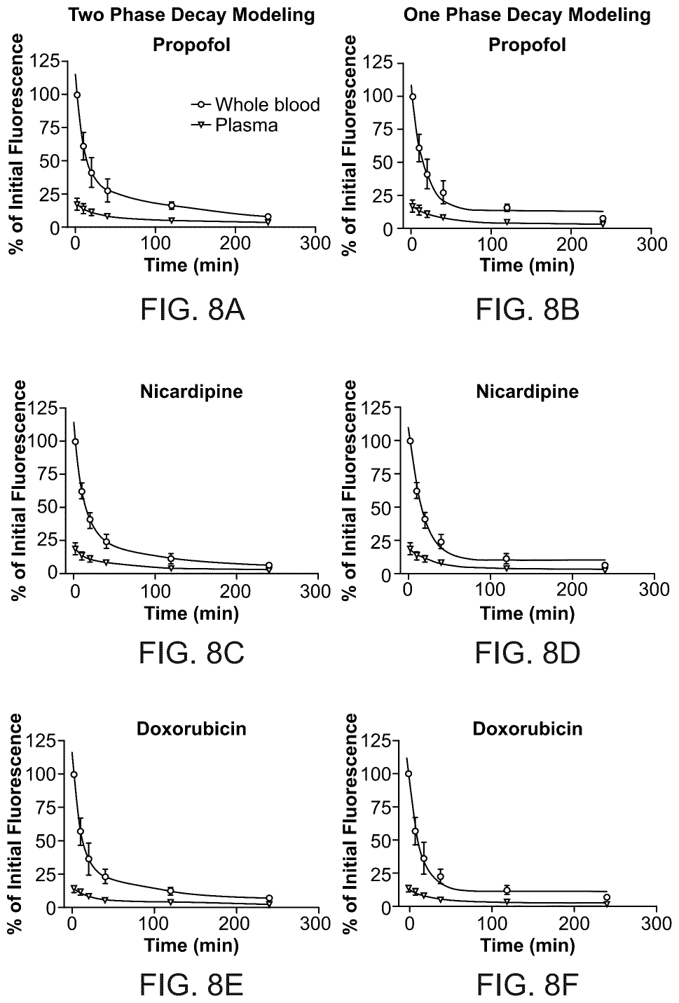

In vivo nanoparticle characteristics. To determine the clearance kinetics, biodistribution, and biocompatibility of the particles in rats, in addition to the indicated drug, the particles were doped with a dye whose infrared fluorescence is quantitative in vivo and in blood samples, and which in free form clears from the blood pool within 3-5 min. For this analysis, among the drugs with substantial loading (FIG. 2), drugs with high (nicardipine), intermediate (propofol), and low (doxorubicin) Log P were chosen. To assess particle kinetics, the fluorescence was quantified for whole blood and plasma samples collected at several time points over hours. The difference between the whole-blood and plasma sample fluorescence indicated the nanoparticle blood concentration. The plasma fluorescence indicated the rate of generation of drug-loaded micelles as the volatile PFP diffuses out of the nanoparticle core. There was no substantial effect of the encapsulated drug on particle kinetics or biodistribution (FIG. 4). For each drug, the particle blood pool concentration followed a dual exponential clearance profile, with half-lives of 10-12 min for a redistribution phase and 77-97 min for an elimination phase (See FIGS. 8a-8f). Based on this profile, a bolus plus infusion protocol was determined to yield a steady blood particle concentration to enable prolonged usage. Indeed, with this bolus plus infusion protocol, a steady blood pool particle concentration was seen for over 40 min, with correspondingly rapid elimination following the halt of infusion (FIG. 4b). Independent of the loaded drug, the particles showed uptake at 24 h primarily in the liver, followed by spleen and lung, with minimal uptake in kidney and heart, and notably no binding to the brain (FIG. 5a-c). In the presently described experiments, 86 rats have received the current formulation of these particles, with some receiving up to nine doses over several weeks, and none have shown visible evidence of toxicity due to particle administration or uncaging. Indeed, no negative change was seen in animal body weight across weeks of multiple nanoparticle administrations (FIG. 5c). These results indicate that, independent of the choice of drug loaded, these nanoparticles are well tolerated and have clinically practical clearance kinetics to enable acute ultrasonic drug uncaging therapies.

The in vivo organ distribution of propofol-loaded PFP/PCL2k-PEG2k nanoparticles in rats, 24 hours after i.v tail-vein injection (n=3) versus untreated rats serving as a negative control was assessed. In some embodiments, nanoparticles accumulated primarily in the liver and spleen, although accumulation was also seen in the lungs, kidneys and heart, to a lesser extent. Notably, there was no accumulation in the brain (without pulsing with ultrasound), which is explained by the exclusion provided by the blood-brain barrier. FIG. 5a: Sample images of IR dye fluorescence in organs harvested 24 h after saline (top) and nanoparticle (bottom) administration to rats. FIG. 5b: Tissue distribution of propofol, nicardipine, or doxorubicin-loaded nanoparticles 24 h after i.v bolus (1 mg/kg encapsulated drug). FIG. 5c: Body weight of rats administered 3 boluses of propofol-loaded nanoparticles over 8 days. Mean+/−S.D. are presented for groups of N=3.

### Example 8

### Efficacious Ultrasonic Drug Uncaging in the Nervous System

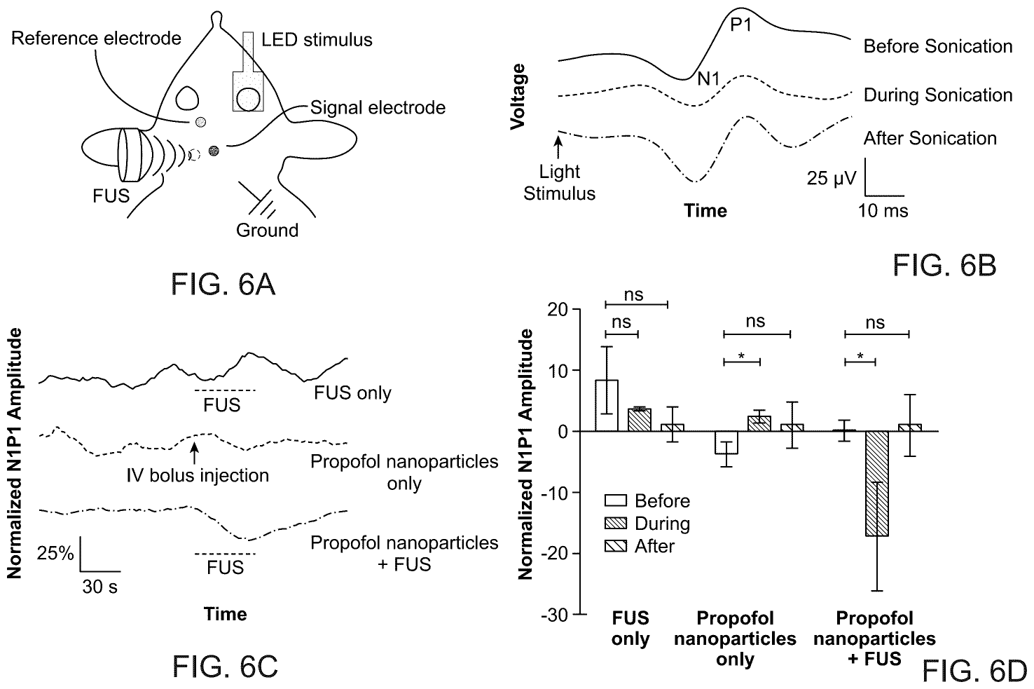

The presently described nanoparticles retained a similar efficacy in vivo as was shown in the embodiments described above. The response of visual evoked potentials (VEPs), an electrophysiological assay of brain function, to ultrasonic propofol uncaging in rat primary visual cortex (V1) was assessed. Light flashes (10 ms@1 Hz) were administered to one eye of a rat (FIG. 6a). A 650 kHz focused ultrasound transducer was coupled to target the contralateral V1. Electrophysiological potentials in response to the light flashes were recorded by a skull electrode placed near midline over the occipital cortex, with a reference electrode in the rostral frontal bone. Each rat underwent the same protocol in which, after the recorded potentials reached a threshold signal amplitude (N1P1 amplitude>60 μV), FUS was applied (60×50 ms pulses, 1 Hz pulse repetition frequency, 1.2 MPa estimated peak in situ pressure) to V1 without nanoparticles in circulation, while recording VEPs. Then, while recording VEPs, propofol-loaded nanoparticles were administered intravenously as a bolus. Then, after waiting at least 10 min from nanoparticle administration to allow redistribution (FIG. 4a), the same FUS protocol was applied to V1. A substantial reduction in the N1P1 VEP amplitude was noted with sonication with nanoparticles in circulation (FIG. 6b-d), indicating an effective anesthesia induced by ultrasonic propofol uncaging. Notably, FUS alone had no similar effect. Nanoparticle administration did not yield such a decrease, and instead induced a slight increase in VEP amplitude, presumably due to a net stimulatory effect of bolus fluid administration. Further, the anesthetic effect of ultrasonic propofol uncaging was limited by the time period of ultrasound application, with anesthesia onset commencing with FUS onset, and recovery commencing with FUS offset (FIG. 6c). In some embodiments, seizure activity could be knocked out in rats, and thus, the system described herein was found to be effective for ultrasonic drug uncaging to attenuate pathologic brain activity. These results confirm that the presently described system is useful for effective ultrasonic propofol uncaging to reversibly knock out physiological brain activity.

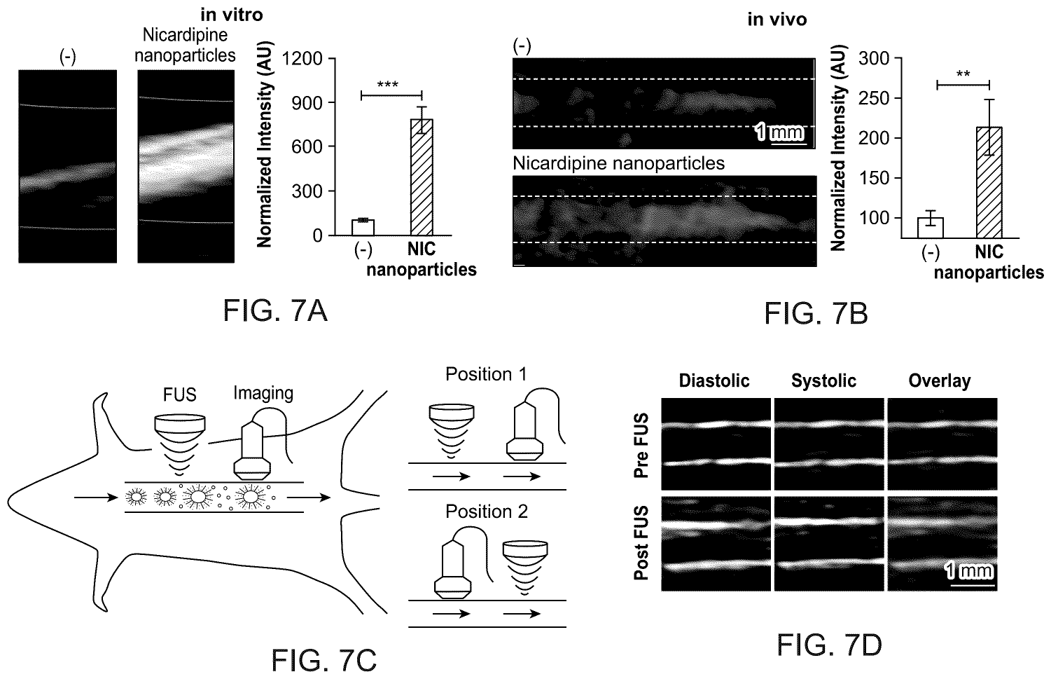

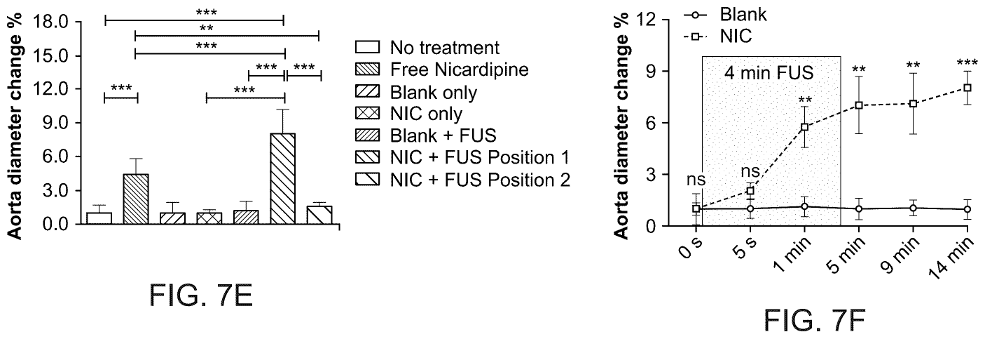

Efficacious ultrasonic contrast enhancement and drug uncaging in the cardiovascular system: The acoustic impedance of PFP at 37° C. was noted to be 0.67 MRayl, and that of blood or soft tissue was typically 1.6-1.7 MRayl. This difference in acoustic impedance suggested that polymeric PFP nanoemulsions should be echogenic in the body, and therefore could serve as ultrasound contrast agents. Indeed, both in vitro (FIG. 7a) and in vivo (FIG. 7b), it was observed that the presence of PFP nanoparticles substantially increased power Doppler ultrasound signals, allowing for more sensitive vascular imaging.

Next, it was noted that in the ultrasonic propofol uncaging experiments (FIG. 6), the uncaged drug had the opportunity to enter into and act upon the brain parenchyma across the blood-brain barrier during the whole time it traversed the capillary bed (order of seconds). It was then assessed whether ultrasonic drug uncaging could measurably affect physiological systems after uncaging in larger vessels with faster flows, where there is not as much time for the critical drug-receptor interaction. Vasodilating agents, such as nicardipine and verapamil are used clinically to relieve arterial spasm as seen with cerebral vasospasm and other conditions, by relaxing the smooth muscle of the vessel wall. For example, nicardipine has been shown to relax the wall of the aorta and increase its distensibility in humans. While effective, these agents have undesirable side effects of generalized hypotension when given systemically, due to decreasing the systemic vascular resistance by action beyond the target vessel. This hypotension can result in end organ infarction in severe cases. In order to minimize this effect, the vasodilator must be infused via an invasive intra-arterial catheter placed within the target vessel or immediately upstream. For ultrasonic vasodilator uncaging to achieve similar effects, the vasodilator must bind the arterial smooth muscle immediately after ultrasound-induced release from the nanoparticles, given that arterial velocities are generally on the order of 0.3-0.5 m/s. It was then assessed whether ultrasonic nicardipine uncaging could yield a visible change in vessel wall compliance of the rat abdominal aorta (˜7 cm in length, ˜1 mm luminal diameter), indicated by the change in vessel diameter over the cardiac cycle. With ultrasonic nicardipine uncaging in the aorta upstream of an ultrasound imaging probe, a substantial difference in systolic vs. diastolic aortic diameters was noted compared to the pre-uncaging baseline (FIG. 7c-f). Confirming its specificity, this effect was not seen with ultrasound alone, with nanoparticle administration alone, with ultrasound applied to blank nanoparticles, or with nicardipine uncaging applied downstream of the imaging probe (FIG. 7e). In fact, compared with a systemic bolus of free nicardipine that is matched in terms of the total nicardipine dose, ultrasonically uncaged nicardipine had a more potent effect on the vessel wall distensibility, even though only a minority of nanoparticles were exposed to the sonication field (FIG. 7e). This confirms that ultrasonic drug uncaging yields a relatively restricted volume of distribution of the drug that is confined to the sonicated region, which in turn results in an effective amplification of the local drug effect. Furthermore, these results demonstrate that this localized drug-receptor binding can occur even in the presence of rapid aortic flows. Notably, the majority of this effect occurred with the first minute of sonication (FIG. 7f), confirming the rapid temporal kinetics of ultrasonic drug uncaging.

FIG. 6|Ultrasonic propofol uncaging reversibly anesthetizes the visual cortex. 6a, Experimental schematic for measuring rat visual evoked potentials (VEPs). 6b, Averaged VEP waveforms before, during, and after sonication (650 kHz, 60×50 ms pulses at 1 Hz pulse repetition frequency, 1.2 MPa est. peak in situ pressure) in animals with propofol-loaded nanoparticles in circulation. 6c, VEP N1P1 amplitude across time. 6d, Average N1P1 amplitude during a 60 s epoch before, during, or after sonication (FUS only and Propofol nanoparticles+FUS groups) or nanoparticle administration (Propofol nanoparticles only group). Mean+/−S.D. are presented for groups of N=3. ns: not significant, *: p<0.05 by two-tailed t-test.

FIG. 7|Ultrasonic nicardipine uncaging locally increases vessel compliance. Nicardipine-loaded nanoparticles increase power Doppler ultrasound signal 7a, in vitro and 7b, in vivo. 7c, Experimental schematic to test if ultrasonic nicardipine uncaging increases rat aortic wall compliance. Uncaging is applied to the aorta either upstream (Position 1) or downstream (Position 2) of imaging. 7d, Ultrasound images of the rat abdominal aorta during systole and diastole, before and after ultrasonic nicardipine uncaging (650 kHz, 240×50 ms pulses at 1 Hz pulse repetition frequency, 1.55 MPa est. peak in situ pressure); Overlay, green=diastolic, red=systolic, yellow=green/red overlap. Averaged rat abdominal aortic diameter at 7e, 14 min after uncaging or 7f, across time, normalized by the initial (0 s) values. FUS=focused ultrasound application, NIC=nicardipine-loaded nanoparticles. Free nicardipine and NIC administered to total drug dose of 134 μg/kg i.v. Mean+/−S.D. are presented for groups of N=3 (7a, 7b) and N=5-6 (7e, 7f). ns: not significant, **: p<0.01; ***: p<0.001 by two-tailed Student's t-tests between the nicardipine-loaded nanoparticles and the corresponding negative conditions (7a, 7b, 7f) or ANOVA and Tukey post-hoc tests (7e, F(6,30)=28.49).

### Example 9

### Nanoemulsion Stability at Varied Temperatures

Propofol-load nanoemulsions were used to assess the particle stability at different temperatures. Z-average size, polydispersity index and free propofol content in the nanoemulsion were evaluated during frozen storage at −80° C. and at 0° C. after thaw. The nanoemulsion was stored at −80° C. after production and the sample was assessed after the 7, 15 and 30 days in storage. The nanoemulsion was then slowly thawed at room temperature and placed on ice. The nanoemulsion was assessed at 45 min and 3 hrs after thawing. The effect of the concentration of nanoemulsion (as indicated by propofol concentration) on particle stability during storage was also studied. The initial concentration of propofol in the nanoemulsion was selected as either 0.5, 1 and 3 mg/ml by adjusting the resuspension PBS volume in nanoemulsion production. Finally, whether repeated freeze-thaw treatment impacts the integrity of nanoemulsion was assessed. The nanoemulsion was thawed as described above and then frozen shortly after sampling. Five cycles were performed consecutively. Propofol-loaded nanoemulsions are stable across multiple freeze-thaw cycles.

Polymeric PFP nanodroplets are herein described and shown to be a versatile platform for ultrasonic drug uncaging, with a ready path for clinical translation. Scalable production methods that are cGMP-compatible and which produce particles that are stable for both long-term frozen storage and for hours of use after thawing are herein described (FIG. 1). Longer hydrophobic blocks of the emulsifying polymer were confirmed to yield greater drug loading, with minimal effect of the choice of polymer on drug uncaging efficacy (FIG. 2). Thus, the ability of this technology to encapsulate and selectively uncage drugs of varying degrees of hydrophobicity was explicitly demonstrated, and that span multiple drug classes and receptor binding profiles (FIG. 3). Indeed, the clearance kinetics and biodistribution of these nanoparticles appears to be independent of the particular drug that is encapsulated (FIGS. 4 and 8). Finally, the utility of ultrasonic drug uncaging was demonstrated to yield potent localized pharmacological bioeffects both in the brain (FIG. 6) and in the body (FIG. 6). Specifically, it was shown that ultrasonic propofol uncaging can yield a potent anesthesia of the visual cortex with sonication (FIG. 6). It was also shown that these are theranostic particles, as they act both as ultrasound contrast agents for vascular imaging (FIG. 7a,b) and may yield focal vessel wall relaxation with ultrasonic nicardipine uncaging that is spatially restricted to the target vessel, even in the setting of rapid aortic flows (FIG. 7c-f).

There are myriad potential applications for ultrasonic propofol uncaging. In the brain, focal uncaging of neuromodulatory agents could allow pharmacological adjuncts to talk or exposure psychiatric treatments that are tailored to the particular neural circuit pathophysiology for a given patient. This technology could also allow pharmacological mapping of neural circuits to better target more permanent interventions such as surgical resection, ablation, or deep-brain stimulation. In the cardiovascular system, treatment of spasm disorders, such as the cerebral vasospasm that unfortunately accompanies many cases of subarachnoid hemorrhage, is difficult given that the agents that best relieve the spasm also act systemically as potent anti-hypertensives. Local relaxation of the walls of the affected vessels, as modeled in FIG. 7, without loss of systemic vascular resistance and therefore systemic hypotension, would be enormously beneficial. Finally, many chemotherapeutics are known to be effective for treatment of a given tumor yet cannot be administered in effective doses systemically due to intolerable side effects in the rest of the body. Ultrasonic chemotherapeutic uncaging within the tumor and its immediate margin is therefore of great utility.

Future work with this technology will move ultrasonic drug uncaging to clinical practice, first by validating this approach in large animal models, and then beginning first-in-human trials to establish the safety and drug uncaging efficacy of this technique. Importantly, the constituent components of these nanoparticles-namely the drugs under consideration, PEG, PLGA, and PFP droplets-have each been used in clinical populations with excellent safety profiles, lowering the barrier to translation for these nanoparticles. Additional future work will focus on expanding this technology to include encapsulation of hydrophilic small molecules, as well as larger macromolecules like peptides, antibodies, and nucleic acids. Indeed, given their potential for clinical translation, their ability to uncage a variety of important drugs, and the potent local pharmacological bioeffects they can induce, polymeric perfluoropentane nanoemulsions are poised to have enormous impact both for clinical care as well as a scientific understanding of how pharmaceuticals mediate their effects.

The preceding merely illustrates the principles used in the present disclosure. It will be appreciated that those skilled in the art will be able to devise various arrangements which, although not explicitly described or shown herein, embody the principles of the present disclosure and are included within its spirit and scope. Furthermore, all examples and conditional language recited herein are principally intended to aid the reader in understanding the principles of the disclosure and the concepts contributed by the inventors to furthering the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the appended claims.

1-16. (canceled)

17. A method of delivering a hydrophobic compound that can pass through the BBB to a subject in need thereof the method comprising:
administering intravenously or into the cerebrospinal fluid (CSF) of [a] the subject a polymeric perfluorocarbon nanoemulsion comprising nanoparticles less than 1 micron in diameter, wherein the nanoparticles comprise
an amphiphilic diblock-copolymer of polyethyleneglycol (PEG) complexed with a polymer selected from a polycaprolactone (PCL); a poly(lactide-co-glycolide) (PLGA); and a poly(L-lactic acid) (PLLA); a high vapor pressure liquid core wherein the high vapor pressure liquid is selected from perfluoromethane, perfluoroethane, perfluoropropane, perfluorobutane, perfluorocyclobutane, perfluropentane, and perfluorohexane; and wherein the hydrophobic compound that can pass through the BBB is selected from a therapeutic agent and a contrast agent; and
delivering an ultrasound pulse to the brain or brain vasculature of the subject with a pressure between 0.8 and 1.8 MPa, a burst length of 10-100 ms, and a frequency between 0.2 and 2.0 MHz, resulting in oscillation and/or expansion of the core and release of the hydrophobic compound from the nanoparticles.

18. The method of claim 17, performed in combination an imaging method following focal drug release.

19. (canceled)

20. The method of claim 17, wherein the polymeric perfluorocarbon nanoemulsion further comprises a cryoprotectant of from 1% to 3% glycerin or sucrose.

21. The method of claim 17, wherein the therapeutic agent is selected from propofol, ketamine, nicardipine, verapamil, dexmedetomidine, modafinil, doxorubicin, and cisplatin.

22. The method of claim 17, wherein the therapeutic agent is a vasodilator.

23. The method of claim 17, comprising a contrast agent.

24. The method of claim 17, wherein the therapeutic agent is treatment for a psychiatric disease.

25. The method of claim 17, wherein the therapeutic agent is treatment for pain.

26. The method of claim 17, wherein the therapeutic agent is treatment for cancer.

27. The method of claim 17, wherein the therapeutic agent is treatment for epilepsy.
